(19) United States
(12) Patent Application Publication
(10) Pub. No.: US 2026/0299568 A1
(43) Pub. Date: Oct. 1, 2026

(54) INFORMATION PROCESSING APPARATUS, CHARACTERISTIC PREDICTION METHOD, AND CONTROL PROGRAM

(71) Applicant: KONICA MINOLTA, INC., Chiyoda-ku, Tokyo (JP)

(72) Inventors: Masaru FUSE, Chiyoda-ku, Tokyo (JP); Hiroshi KITA, Chiyoda-ku, Tokyo (JP)

(73) Assignee: KONICA MINOLTA, INC., Chiyoda-ku, Tokyo (JP)

(21) Appl. No.: 19/168,375

(22) PCT Filed: Mar. 26, 2024
(86) PCT No.: PCT/JP2024/011956

§ 371 Date: Jun. 12, 2026

(30) Foreign Application Priority Data
Mar. 31, 2023 (JP) 2023-057129

(51) Int. Cl. G05B 19/418

(52) U.S. Cl. CPC G05B 19/41875; G05B 2219/32194

(57) ABSTRACT

An information processing apparatus 10 includes, in a design and manufacturing process of a product or a material having a plurality of predetermined characteristics: a descriptor construction section 110 that constructs a descriptor based on data obtained by measuring information regarding a structure and/or a property of an object in the design and manufacturing process under a plurality of predetermined measurement conditions and; a predictor 120 that predicts a plurality of characteristics of a final product of the design and manufacturing process based on the descriptor.

### TECHNICAL FIELD

The present invention relates to an information processing apparatus, a characteristic prediction method, and a control program.

### BACKGROUND ART

In order to determine compositions/processes of materials for obtaining desired material characteristics in a transition of manufacturing to a complicated system using, for example, a polymer material, a composite material, and a biomaterial, it is necessary to acquire a large amount of deductive information such as computer simulation data or inductive information such as experimental data, and this leads to a decrease in work efficiency. In addition, it is difficult to acquire, by simulation or the like, information regarding characteristics based on human sensitivity and preference.

A system for estimating kinetics disclosed in Patent Literature 1 acquires multivariate experimental data regarding an oxidation-reduction potential using three working electrodes different in material or surface treatment for a culture solution serving as an object to be measured, and estimates optimization conditions for the object to be measured using the acquired data.

Non Patent Literature 1 indicates that a concept called Process-Structure-Property Linkage has been introduced for the development of composite materials (thermal diffusion films) obtained by adding inorganic fillers to resins to improve development efficiencies.

In the development and production of a film that contains fillers for heat diffusion, a mechanism between both the composition of materials used and process conditions (Process) which are explanatory variables (input) and heat diffusion properties (Property) which are objective variables (output) is complicated. Therefore, an inductive verification by an experiment or the like requires a large amount of data, and a deductive verification by a simulation or the like not only requires much time and cost but also may not find a suitable solution depending on an assumed model.

To address such a problem, in the method disclosed in Non Patent Literature 1, a “filler particle dispersion structure” (Structure) is deductively derived from a process (Process) by computer simulation as a descriptor linking an explanatory variable (Process) and an objective variable (Property) without directly associating them, each of the relation between Process and Structure and the relation between Structure and Property is converted into a verifiable relation, and the results are connected, by which the efficiency in the input/output design (Process-Property) of a composite material is increased. Furthermore, a model of computer simulation is set to have a cubic mesh configuration and a simulation at a scale (factor) corresponding to a characteristic to be realized or a notable performance is performed, whereby efficient verification using an optimum descriptor is enabled.

### CITATION LIST

### Patent Literature

Patent Literature 1: JP 2021-43097 A

### Non Patent Literature

Non Patent Literature 1: Shinji Ozawa (Kaneka Corporation), “Research and Development of Resin-Inorganic Filler Composite Materials”, [online], Jan. 19, 2022, Ultra-Advanced Materials Ultra-High Speed Development-Based Technology Project (Ultra-Ultra PJ) Final Results Report Meeting, Ultra-Advanced Materials Ultra-High Speed Development-Based Technology Project (Ultra-Ultra PJ), Internet <URL https://www.admat.or.jp/library/5975666db3de4b020a7803ae/61ea4ddf3b19fb676e934836.pdf>

### SUMMARY OF INVENTION

### Technical Problem

However, in the method disclosed in Non Patent Literature 1, the descriptor is deductively derived from starting materials and process conditions by using computer simulation or the like, and thus, the method has a problem that the calculation cost increases as the physical properties and the like of a final product become more complicated and complex. Furthermore, depending on the assumed model, the relation between the descriptor and the objective variable may not be valid, and thus a solution may not be derived. In other words, an appropriate model is required for simulation, which is a deductive method, and it is not possible to eliminate the dependency on personal skills such as know-how related to the design.

Furthermore, in order to perform simulations on different scales corresponding to a plurality of characteristics, an enormous amount of calculation power is required, and it is difficult to immediately obtain results. There may be a problem that simulation of an appropriate scale cannot necessarily be executed for a characteristic to be realized. As described above, the descriptor based on the simulation cannot be a decisive solution in achieving high development efficiency by accelerating the experiment-verification cycle.

The present invention has been made in consideration of the above-described circumstances, and in a design or manufacturing process (hereinafter, referred to as a design and manufacturing process) of designing or manufacturing a material, a product, or the like having a plurality of characteristics at the same time, the present invention simultaneously acquires data pieces regarding structures and properties in a region from a micro region to a macro region under measurement conditions or by measurement means with different scales for a final product output as a result of the process, an intermediate product between a starting material and the final product generated in the middle of the process, and the starting material, and uses data obtained by linking the data pieces as a new descriptor. An object of the present invention is to appropriately predict a plurality of characteristics to be realized during the manufacture of food or the like by a deductive argument based on complicated object manufacturing, human sensitivity, and the like by using the descriptor, and to make a design and manufacturing process efficient.

### Solution to Problem

The above object of the present invention is achieved by the following means.

(1) An information processing apparatus including, in a design and manufacturing process of a product or a material having a plurality of predetermined characteristics:
a descriptor construction section that constructs a descriptor based on data obtained by measuring information regarding a structure and/or a property of an object in the design and manufacturing process under a plurality of predetermined measurement conditions; and
a predictor that predicts a plurality of characteristics of a final product of the design and manufacturing process based on the descriptor.

(2) The information processing apparatus according to (1), wherein the object is a final product obtained as an output result of the design and manufacturing process, an intermediate product generated during the design and manufacturing process, and/or a starting material input to the design and manufacturing process.

(3) The information processing apparatus according to (1), wherein the plurality of measurement conditions includes at least a measurement condition in which spatial or temporal measurement regions and/or spatial or temporal measurement scales are different.

(4) The information processing apparatus according to (3), wherein the plurality of measurement conditions is selected depending on the predetermined characteristics of the object.

(5) The information processing apparatus according to (3), wherein the plurality of measurement conditions includes at least a first measurement condition having a predetermined first measurement region and a first measurement scale, and a second measurement condition set based on the first measurement condition, and
the second measurement condition has a second measurement region smaller than the first measurement region or a second measurement scale smaller than the first measurement scale.

(6) The information processing apparatus according to (5), wherein one of data acquired under the first measurement condition and data acquired under the second measurement condition is information regarding the structure of the object, and the other is information regarding the property of the object.

(7) The information processing apparatus according to (3), wherein measurement timings under the plurality of measurement conditions are simultaneous with each other or have a certain temporal relation with each other.

(8) The information processing apparatus according to (7), wherein the measurement timings are determined to be synchronized with a change in position or a change in place of the object in the design and manufacturing process as the certain temporal relation.

(9) The information processing apparatus according to (5), wherein, under the first and second measurement conditions, the first and second measurement regions have a predetermined spatial relation with each other.

(10) The information processing apparatus according to (1), wherein the predictor predicts a plurality of characteristics of the final product from the entire or a part of the descriptor, using a first trained model that outputs characteristics of the object based on the descriptor.

(11) The information processing apparatus according to (1), further including a process parameter adjuster that, using a second trained model that outputs a parameter related to the design and manufacturing process based on the descriptor, adjusts the parameter based on the entire or a part of the descriptor.

(12) The information processing apparatus according to (3), wherein the plurality of measurement conditions uses frequencies and/or signal lengths different from each other.

(13) The information processing apparatus according to (12), wherein the plurality of measurement conditions is executed by a plurality of measurement means, and
the plurality of measurement means includes at least any two of an acoustic sensor, an electromagnetic-wave sensor, and a light emission sensor.

(14) A characteristic prediction method including, in a design and manufacturing process of a product or a material having a plurality of predetermined characteristics:
a step (a) of constructing a descriptor based on data obtained by measuring information regarding a structure and/or a property of an object in the design and manufacturing process under a plurality of predetermined measurement conditions; and
a step (b) of predicting a plurality of characteristics of a final product of the design and manufacturing process based on the descriptor.

(15) A control program for causing a computer to execute processing including, in a design and manufacturing process of a product or a material having a plurality of predetermined characteristics:
a step (a) of constructing a descriptor based on data obtained by measuring information regarding a structure and/or a property of an object in the design and manufacturing process under a plurality of predetermined measurement conditions; and
a step (b) of predicting a plurality of characteristics of a final product of the design and manufacturing process based on the descriptor.

### Advantageous Effects of Invention

An information processing apparatus according to the present invention includes, in a design and manufacturing process of a product or a material having a plurality of predetermined characteristics: a descriptor construction section that constructs a descriptor based on measurement data obtained by measuring information regarding a structure and/or a property of an object in the design and manufacturing process under a plurality of predetermined measurement conditions; and a predictor that predicts a plurality of characteristics of a final product of the design and manufacturing process based on the descriptor. Accordingly, it is possible to accurately predict a plurality of characteristics of a final product by using an AI technology, a statistical analysis technique, or the like, to optimize parameters of a design and manufacturing process, and to perform design or manufacturing with high efficiency.

### BRIEF DESCRIPTION OF DRAWINGS

FIG. 1 is a diagram illustrating a configuration of a design and manufacturing process including an information processing apparatus 10 and the like according to a first embodiment.

FIG. 2A is a schematic diagram for conceptually describing a scalable descriptor.

FIG. 2B is a schematic diagram for conceptually describing a scalable descriptor corresponding to a constituent size of a final product.

FIG. 3 is a schematic diagram for conceptually describing the flow of data in the information processing apparatus.

FIG. 4 is a schematic diagram for describing the structure of a scalable descriptor.

FIG. 5 is a schematic diagram for describing a method for constructing a scalable descriptor.

FIG. 6 is a diagram illustrating a configuration of a design and manufacturing process including an information processing apparatus 10b and the like according to a second embodiment.

FIG. 7 is a diagram illustrating a configuration of a design and manufacturing process including an information processing apparatus 10c and the like according to a third embodiment.

FIG. 8 is a flowchart illustrating processing of predicting characteristics and processing of adjusting process parameters executed by the information processing apparatus 10 or 10c.

FIG. 9 is a flowchart illustrating a process of a machine learning method for a first trained model.

FIG. 10 is a flowchart illustrating a process of a machine learning method for a second trained model.

FIG. 11 is a flowchart illustrating processing of predicting characteristics and processing of adjusting process parameters executed by the information processing apparatus 10b.

FIG. 12 is a flowchart illustrating a process of a machine learning method for a third trained model.

FIG. 13 is a flowchart illustrating another processing of predicting characteristics and another processing of adjusting process parameters executed by the information processing apparatus 10 or 10c.

FIG. 14 is a flowchart illustrating a process of a machine learning method for a fourth trained model.

FIG. 15 is a flowchart illustrating a process of a machine learning method for a fifth trained model.

FIG. 16 is a diagram illustrating a configuration of a design and manufacturing process in Example 1.

FIG. 17 is a diagram illustrating a configuration of a design and manufacturing process in Example 2.

FIG. 18 is a diagram illustrating a configuration of a design and manufacturing process in Example 3.

FIG. 19 is a diagram illustrating a configuration of a design and manufacturing process in Example 4.

FIG. 20 is a diagram illustrating a configuration of a design and manufacturing process in Example 5.

FIG. 21 is a diagram illustrating a configuration of a design and manufacturing process in Example 6.

FIG. 22 is a diagram for describing a luminescent dye molecule in a first sensing device.

FIG. 23 is a diagram for describing a production dye molecule of a luminescent dye molecule.

FIG. 24 is a flowchart illustrating an analysis method using the luminescent dye molecule.

FIG. 25 is a schematic cross-sectional view of a second sensing device.

FIG. 26 is a diagram for describing another example of the second sensing device, in which (a) is a plan view of a sensing element 1, and (b) is a schematic cross-sectional view taken along a line A-A′.

FIG. 27 is a diagram illustrating another second sensing device, in which (a) is a schematic plan view, and (b) is a schematic cross-sectional view taken along a line A-A′.

FIG. 28 is a schematic diagram illustrating a configuration of a sensing system relating to the second sensing device.

### DESCRIPTION OF EMBODIMENTS

Embodiments of the present invention will be described below with reference to the accompanying drawings. It is to be noted that the scope of the present invention is not limited to the disclosed embodiments. Note that in the description of the drawings, the same components are denoted by the same reference signs, and redundant descriptions will not be repeated. In addition, dimensional ratios in the drawings are exaggerated for convenience of description and may be different from actual ratios.

In the following description, an object from which measurement data is acquired includes a final product manufactured in a design and manufacturing process, an intermediate product generated in the middle of the design and manufacturing process, and a starting material input to the design and manufacturing process. In the following, the final product, the intermediate product, and/or the starting material may be referred to as an object.

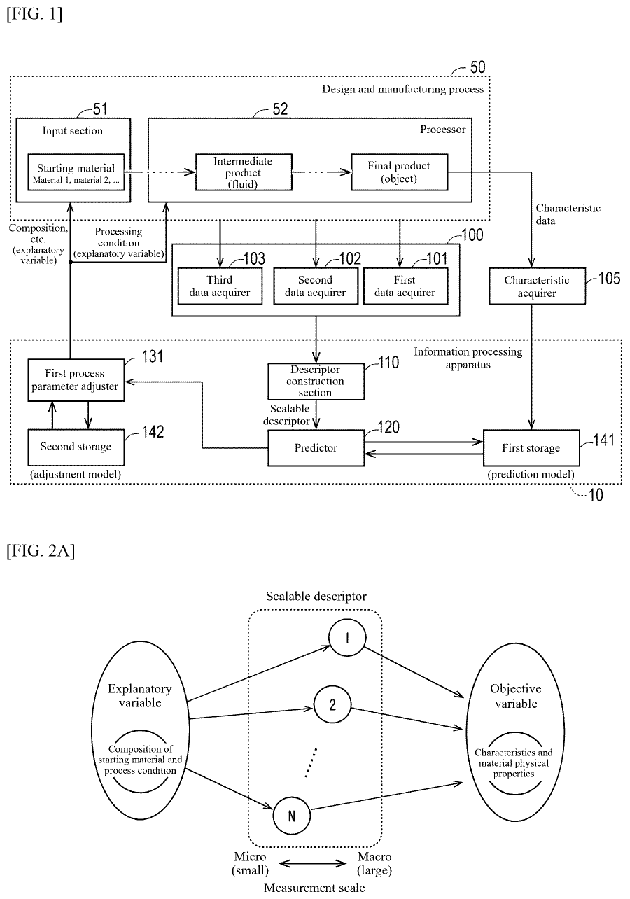

FIG. 1 is a diagram illustrating a schematic configuration of a design and manufacturing process including an information processing apparatus 10 according to a first embodiment. The information processing apparatus 10 includes a stand-alone personal computer or an on-premise server installed in a site such as a factory in which the design and manufacturing process 50 is provided, a cloud server using a commercial cloud service, or the like. The information processing apparatus 10 includes a CPU, a RAM, a storage, a communication I/F, and the like. The information processing apparatus 10 acquires a scalable descriptor and data (hereinafter, referred to as characteristic data) regarding characteristics and/or physical properties to be realized of a final product via an acquirer 100 and a characteristic acquirer 105 (both of which will be described later) that are separately provided. The scalable descriptor is a descriptor indicating a structure (that is, a structure of the object) and/or a property or the like (hereinafter, referred to as a structure of the object and the like) of a final product, an intermediate product, and/or a starting material from the design and manufacturing process 50. Then, the information processing apparatus 10 predicts the characteristic and the like of the final product based on the structures and/or properties, and adjusts parameters such as the composition of the starting material in the design and manufacturing process 50 and the processing conditions of processes from the starting material to the final product. Here, the characteristic and the like of the final product include at least one of physical properties, quality, and functions of the product. Furthermore, the characteristic and the like of the final product may include, in addition to quantifiable properties such as mechanical properties, physical properties, thermal properties, electrical properties, and combustibility, at least one of difficult-to-quantify properties at present such as taste, texture, and touch. Note that in the following, the wording “information processing apparatus and the like” refers to the acquirer 100, the characteristic acquirer 105, and the information processing apparatus 10.

Here, when the final product is obtained from a starting material through a predetermined process, the intermediate product is a substance obtained in the middle of the process. Alternatively, the intermediate product is a substance that is produced in an intermediate step during the production of the final product and is different from the starting material and the final product. The intermediate product includes not only a compound synthesized in the middle of production of a generally defined chemical product but also a substance or the like generated or output in a series of steps during the production of things in a broad sense. For example, the intermediate product is a culture solution containing cells or microorganisms during bio-production, a resin in a molten state kneaded with a filler in the production of a composite fiber-reinforced resin material, a fluid material under flow synthesis in the production of a chemical synthesis product, or the like. Note that the process may include not only a single process but also a plurality of processes.

In addition, the object is, for example, a compound in a solid phase, a liquid phase, or a gas phase, or a mixture thereof. The mixture is, for example, a solution obtained by dissolving a certain starting material in another liquid (starting material).

The acquirer 100 includes, for example, a plurality of data acquirers. Each data acquirer includes various sensors and measuring instruments having different measurement scales, and measures data regarding the structure and the like of the object. FIG. 1 illustrates an example in which the acquirer 100 includes three data acquirers, but the acquirer 100 is not limited thereto. It is sufficient that the acquirer 100 includes a plurality of data acquirers, and the number of the data acquirers may be two, three, or more. In addition, all of the plurality of data acquirers does not necessarily have measurement scales different from each other, and even if the measurement scales are similar to each other or overlap each other, the data acquirers may have measurement ranges different from each other. The information processing apparatus 10 stores the scalable descriptor and the characteristic and the like of the final product acquired by the characteristic acquirer 105. Then, the information processing apparatus 10 predicts the characteristic and the like of the final product from the scalable descriptor and outputs appropriate values of parameters related to the composition of the starting material and the processing conditions of the process from the starting material to the final product.

The design and manufacturing process 50 includes an input section 51 to which a starting material is input, and a processor 52 that performs a predetermined process on the input starting material to generate a final product. In the design and manufacturing process 50, a treatment for high-concentration lysates such as culturing, brewing, kneading, and doping is performed, and as a result, an intermediate product and a final product are output. Then, the information processing apparatus 10 simultaneously converts the structure and/or the property of the object into data and acquires the data by using a plurality of sensors or the like (hereinafter, referred to as measurement modalities) with the starting material, the intermediate product, and/or the final product as the object.

The design and manufacturing process 50 can be applied to, for example, each type of industry described below, such as “1. Bio-production”, “2. Development of composite materials”, “3. Production of chemical synthesis product”, “4. Food Manufacture”, “5. Cosmetic manufacture”, “6. Pharmaceutical manufacturing”, and “7. Treatment of waste liquid after manufacturing of products” (hereinafter, also referred to as applicable types of industry 1 to 7). That is, the applicable types of industry to which the information processing apparatus and the like according to the present embodiment are preferably used and an object from which a scalable descriptor used for prediction of characteristics of a final product (output matter) at that time is acquired include the following.

### 1. Bio-Production

Bio-production is an attempt to perform efficient manufacturing based on natural or biologically derived resources (biomass) by utilizing a biochemical process typified by photosynthesis instead of a conventional chemical process. In the production of materials, fuels, and chemicals or the culture of small and dynamic useful varieties such as microorganisms (hereinafter, such production and culture are referred to as bio-production) with such biotechnology, the production process and the product are more complex as compared with the conventional chemical synthesis products, and in addition, the physical properties and the process are not necessarily elucidated deductively in some cases. For this reason, it is difficult to assess the quality of the final product in the bio-production. In the present embodiment, the structures, properties, and the like of a starting material, an intermediate product, and/or a final product are acquired as multidimensional and multivariate descriptors with a high throughput, and appropriate and quick process control is implemented.

### 2. Development of Composite Materials

The introduction of lightweight plastics, resin materials, and the like is also progressing in areas where strength and rigidity are required and where metals have been used so far. In addition, properties required for materials are becoming more sophisticated and complex, and accordingly, the materials themselves are also changing to become more complex. For example, in a composite material which has been increasingly used for a structure or the like due to having both lightweight properties and high rigidity, a type in which long fiber-based prepregs are stacked and compressed has been conventionally a mainstream. However, due to a problem of a manufacturing cost, complication of a product shape, and the like as a background, products have appeared that are obtained by subjecting pellets that have been heated and melted to injection molding, the pellets being obtained by kneading a thermoplastic resin and a short fiber filler. However, this manufacturing method has a risk that the strength of the products is greatly reduced due to a weld line generated when the injected resin is branched, rejoined, and solidified in a mold, unevenness of the dispersed filler, and the like. As a method for addressing such a problem in terms of process, there is a short-shot method or the like, but it takes a large amount of time and man-hours and is inefficient. In view of this, there is a demand for a method of surely improving the quality of a final product by efficiently acquiring data regarding the state of a molten material to be poured into a mold, the structure of the final product, and the like, and promptly feeding back the result. In the present embodiment, multidimensional and multivariate quality-related data pieces regarding the structures and properties of a starting material, an intermediate product, and/or a final product are evaluated as descriptors, and the results are promptly fed back.

### 3. Production of Chemical Synthesis Product

As a chemical synthesis method, a flow method has attracted attention. The flow method is a production method for performing synthesis with the inside of a vessel being pressurized and a small amount of a reaction liquid being passed. This method is highly efficient and can achieve scale-up. The flow method is considered to be an effective method also as a future production technology of complicated chemical products using naturally derived resources by biotechnology. However, even in this method, it is becoming difficult to simply assess whether the quality is good or not with an increase in complicatedness and complexity of the products and materials as described above. In addition, in order to increase production efficiency, it is necessary to observe not only the state of a final product but also the state of an intermediate product during synthesis and to promptly feed back the result to a production process. However, at present, the mainstream of means that can monitor a substance in a fluid state in real time is a thermometer, a pressure gauge, or a camera used for an appearance (visual) inspection or the like, and sufficient data cannot be acquired in response to a complicated product or material. In the present embodiment, multidimensional and multivariate data pieces which affect manufacturing quality and which relate to a starting material, an intermediate product, and/or a final product in a fluid state are acquired and used as descriptors to perform quick feedback processing.

### 4. Food Manufacture

In recent years, with the realization of a sustainable society as a background, the development of new food materials has been advanced, and for example, some alternative meats are coming onto the market. A food material is an object directly linked to human sensibility and has many evaluation indices that cannot be quantified yet, such as taste, texture, feeling on the tongue, and smooth sensation in the throat. Furthermore, in view of the diversity of materials serving as a base of the food material, the number of parameter sets to be considered at the time of production is astronomically high, and thus, a complex system which cannot be elucidated chemically at all is exhibited. Therefore, the development and production of food materials are dependent on evaluation by humans who actually eat the food materials, and involve many problems in terms of stability, reproducibility, efficiency, and the like. For example, a proteinaceous food material (milk drink, sausage, collagen, or the like) is manufactured by selecting a microorganism, performing transformation, and then, performing cell growth and fermentation in a reactor, recovering a protein, and mixing and synthesizing a plurality of proteins. In the production process of such a proteinaceous food material, it is very important to manage the growth state of microorganisms in a culture solution. At present, various sensors are installed to manage and control the state of the inside of the reactor, but it is difficult for a conventional single-function sensor device to accurately grasp multivariate data such as the state of the microorganisms in the culture solution. In the present embodiment, a food product, the quality of which is difficult to quantify, is thus accurately produced from a complicated material such as a living thing. For such a purpose, in the present embodiment, the states of not only a final product but also a starting material and an intermediate product are converted into multidimensional and multivariate data as descriptors, and are quickly fed back to the manufacturing process.

### 5. Cosmetic Manufacture

Cosmetics are in direct contact with human skin and are required to have particularly high quality. With the diversity of society in recent years, its demand has increasingly expanded, and a wide variety of products have been commercialized in place of the conventional mass production and mass consumption. On the other hand, while corporate responsibility for products tends to be questioned more than ever, the way of thinking about quality assurance and safety is also changing. That is, there is an increasing tendency to emphasize “results” such as characteristics of products instead of a tendency based on “reasons” such as principles and mechanisms, and there is an increasing need for actual inspection of individual products rather than sampling inspection after mass production. Cosmetics are a type of complex system in which various elements are intertwined. However, there is a limit to the parameters that can be acquired in the evaluation and measurement of the final product, and it is difficult to describe the quality of the final product quantitatively or numerically. As a result, the assessment of whether a cosmetic is good or not has to rely on human eyes, the five senses including smell and touch, and ambiguous indicators such as the past experience and sensibility, which is extremely inefficient. On the other hand, most of cosmetics handle a liquid (fluid) as an intermediate product in their design and manufacturing processes. If the state of the intermediate product is evaluated in addition to the final product, it is conceivable that many parameters that describe the product quality can be acquired. However, conventional single-function sensors such as a thermometer, a viscometer, a pH meter, and a camera cannot acquire data of sufficient quantity and quality corresponding to product quality having complexity. In the present embodiment, multidimensional and multivariate data is acquired for the final product, the intermediate product, and the starting material, and these are used as descriptors to achieve quick processing.

### 6. Pharmaceutical Manufacturing (Bio-Pharmaceutical Manufacturing)

Pharmaceuticals act directly on the human body and are naturally required to be of high quality. In recent years, the role that pharmaceuticals play has become increasingly important due to an aging population of developed countries, a worldwide population increase, or a worldwide pandemic expansion. On the other hand, the waves of biotechnology also extend to the pharmaceutical field, and efforts are being made to produce more effective pharmaceutical products from animal cells or the like by applying gene recombination technology or the like in place of conventional chemical synthesis methods. However, as compared with conventional pharmaceutical products of low-molecular compounds, in some cases, the mechanism has not been completely elucidated in the manufacturing process of biopharmaceuticals handling microorganisms and the like. For this reason, the manufacturing process of biopharmaceuticals entails a problem that many more parameters must be monitored and measured. In addition, from the viewpoint of ensuring the quality of pharmaceuticals, sampling inspection of final products has fundamental problems in terms of safety and efficiency, and in the era of biopharmaceuticals, sampling inspection is considered to be an even greater problem. In addition, most of biopharmaceutical processes treat a fluid (culture solution) as an intermediate product leading to a final product. Therefore, if the intermediate product is evaluated in addition to the final product, many parameters associated with the properties of pharmaceuticals are efficiently acquired, and if the evaluation results are fed back, it is highly likely that pharmaceuticals with higher quality can be stably produced. In the present embodiment, multidimensional and multivariate data can be acquired for the starting material, the intermediate product, and the final product, and can be quickly subjected to feedback processing as descriptors.

7. Treatment of Waste Liquid after Manufacturing of Products

In order to break away from the production of petroleum-derived products and realize a circulatory society, innovation in manufacturing has advanced. The reduction in an environmental load regarding a starting material, a manufacturing process, and a final product is an important requirement, and further, how to treat emissions generated during and after manufacturing is also an important issue. Therefore, activated sludge, which has been used for a long time as a method for treating waste liquid or sewage, has attracted attention again. The activated sludge is a collection of microorganisms such as bacteria and protists. Since the state of the activated sludge changes from moment to moment depending on the season and the water treatment condition, continuous monitoring is required, but under the present circumstances, the monitoring is often performed manually by using a microscope or the like, which is not efficient. Further, from the viewpoint of suppressing the generation amount of excess sludge which is said to exceed 100 tons per year, it is essential to suppress the sludge conversion rate and to increase the concentration of organic matter in the mixture in an aeration tank, and it is necessary to precisely control many of these treatment environmental conditions. In the present embodiment, it is possible to acquire multidimensional and multivariate data relating to the state of the microorganisms in a liquid and the environmental conditions thereof, and to perform quick control and processing using the acquired data as a descriptor.

### (Acquirer 100)

The acquirer 100 acquires data indicating the structure and the like of an object. In FIG. 1, the acquirer 100 includes a first data acquirer 101 to a third data acquirer 103 which acquire measurement data regarding the object at different scales. Note that, as described above, all of the three data acquirers do not necessarily have to have different measurement scales, and even if the measurement scales are the same as each other or overlap each other, the data acquirers may have measurement ranges different from each other. The acquirer 100 may include a sensor or a measurement instrument for each data acquirer. Alternatively, the acquirer 100 may be composed of a communication I/F and acquire data from a sensor or the like provided in advance in the design and manufacturing process 50. The measurement data is not limited to numerical data, and may be image data (a still image and/or a moving image) or the like. Note that the positions (numbers) of the data acquirers in FIG. 1 do not define the specific positional relation among the data acquirers in the design and manufacturing process, and the data acquirers are not necessarily arranged in numerical order in the design and manufacturing process. The acquirer 100 acquires, for example, data related to the structures and the like of final products and/or intermediate products of the above-described applicable types of industry 1 to 7.

### (Characteristic Acquirer 105)

The characteristic acquirer 105 acquires data indicating the characteristic and the like of the final product. The characteristic acquirer 105 may include a sensor or a measurement instrument. Alternatively, the characteristic acquirer 105 may be composed of a communication I/F and acquire measured data from a sensor or the like provided in advance in the design and manufacturing process 50. The characteristic acquirer 105 may be configured as an operation input device, and may be configured to receive an input of data acquired in advance.

### (Information Processing Apparatus 10)

The information processing apparatus 10 includes a descriptor construction section 110, a predictor 120, a first process parameter adjuster 131, a first storage 141, and a second storage 142.

The descriptor construction section 110 uses the measurement data acquired by the plurality of data acquirers in the acquirer 100 as input data after mutually defining a certain spatial and/or temporal relation, and constructs a scalable descriptor to be input to the predictor 120 according to a predetermined procedure (described later).

The predictor 120 includes a first trained model and outputs (predicts), using the first trained model, the characteristic and the like of a final product based on the input scalable descriptor. A training method for the first trained model will be described later.

The first process parameter adjuster 131 includes a second trained model and outputs or adjusts the composition and the like of the starting material in the input section 51 and the processing condition in the processor 52 based on the input scalable descriptor by using the second trained model. In the following, the composition and the like and the processing condition are referred to as process parameters. The process parameters include at least one of the composition, concentration, particle size, content, size, shape, and the like of a material serving as a starting material of the final product, and environmental conditions such as temperature, humidity, pressure, pH, applied current, applied voltage, and flow rate in a process of treating the material.

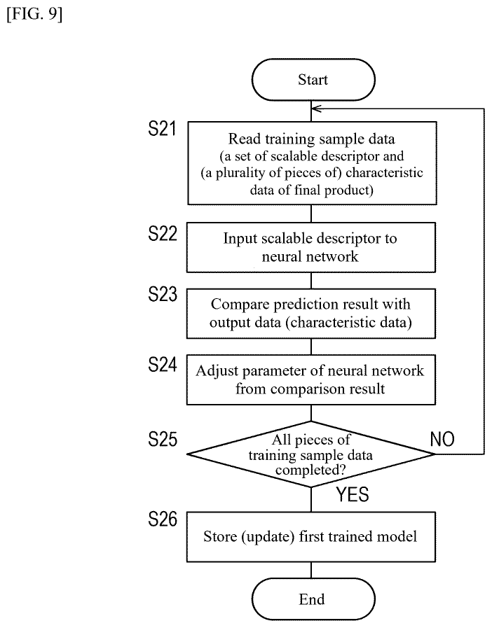

The first storage 141 stores a plurality of sets of data, each of which includes the scalable descriptor constructed by the descriptor construction section 110 and the characteristic and the like of the final product acquired by the characteristic acquirer 105. This is training sample data (training data) and is used for training the first trained model used in the predictor 120. A training method for the first trained model will be described later with reference to FIG. 9.

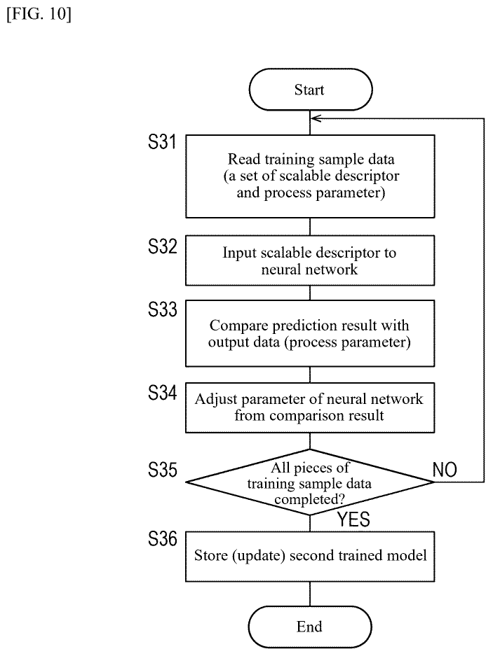

The second storage 142 stores a plurality of sets of data, each of which includes the scalable descriptor constructed by the descriptor construction section 110 and the process parameter input to the input section 51 and/or the processor 52. This is training sample data (training data) and is used for training the second trained model used in the first process parameter adjuster 131. A training method for the second trained model will be described later with reference to FIG. 10.

### (Scalable Descriptor)

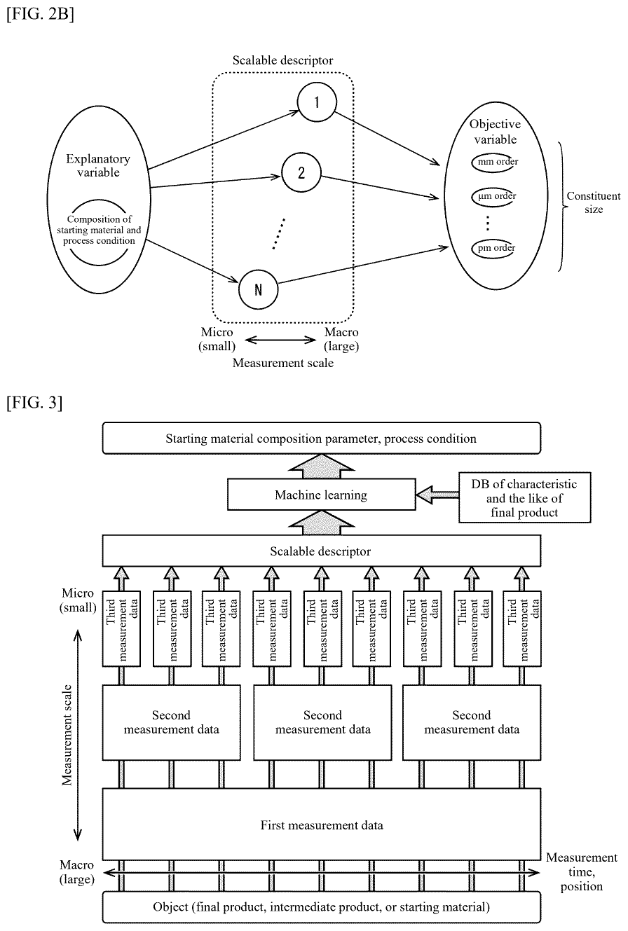

The scalable descriptor will be described below with reference to FIGS. 2A to 4.

FIG. 2A is a schematic diagram for conceptually describing the scalable descriptor. The process parameters (composition of the starting material, process condition, and the like) are used as explanatory variables. The characteristic and the like of the final product (characteristic, material physical property, and the like of the final product) are used as objective variables. A final product and an intermediate product are obtained by the design and manufacturing process 50 according to the process parameters, and scalable descriptors corresponding to the structures and the like of the final product, the intermediate product, and the starting material are obtained.

The scalable descriptor is selected so as to have a predetermined relation with the characteristic and the like to be realized of the final product. The characteristic and the like to be realized of the final product are characteristics required or characteristics to be noted as the final product (hereinafter, also simply referred to as characteristic and the like to be realized). Furthermore, the predetermined relation means that the scalable descriptor and the characteristic and the like have a correlation or coefficient of determination of at least a certain level by statistical analysis or machine learning.

It is highly likely that the characteristic and the like of the final product can be described not only by the structure and the like of the final product but also by the structures and the like of the intermediate product and the starting material. Therefore, in addition to the structure and the like of the final product, the structure and/or the property of the intermediate product and/or the starting material are used as elements of the scalable descriptor, whereby it is possible to more accurately predict the characteristic and the like of the final product. In view of this, an appropriate structure and the like of the intermediate product and/or starting material can be selected in accordance with the characteristic and the like to be realized of the final product.

FIG. 2B is another schematic diagram for describing the scalable descriptor. As illustrated in FIG. 2B, the scalable descriptor includes a plurality of elements (measurement data) corresponding to constituent sizes or unit structures (hereinafter, referred to as constituent size or the like) which determine or are main factors for determining the characteristic and the like to be realized of the final product. An appropriate element can be selected from the scalable descriptor depending on the characteristic and the like to be realized. Here, the constituent size or the like that determines the characteristic and the like to be realized includes, for example, an interatomic distance, an intermolecular distance, a unit cell, a particle diameter of an inclusion, an aggregation size, and an element size.

The final product may simultaneously have a plurality of characteristics and the like to be realized in the final product. In this case, the scalable descriptor includes a plurality of elements (measurement data) of a scale corresponding to a constituent size corresponding to the characteristics and the like to be realized, and a descriptor of an appropriate scale can be selected for each characteristic and the like to be realized. For example, if the characteristic and the like to be realized of the final product relate to an elastic characteristic or structural information, the constituent size is in a range from the order of millimeters to the order of micrometers, and measurement data of a scale corresponding thereto is included as an element of the scalable descriptor. Furthermore, if the characteristic and the like to be realized relate to conductivity or dielectricity, measurement data of a scale corresponding to a constituent size on the order of micrometers or less is included as an element of the scalable descriptor. In addition, in a case where the characteristic and the like to be realized relate to an optical characteristic such as a refractive index, an atomic force/molecular force, or the like, measurement data of a scale corresponding to a constituent size in a range from the order of nanometers to the order of picometers is included as an element of the scalable descriptor.

### (Procedure of Constructing Scalable Descriptor)

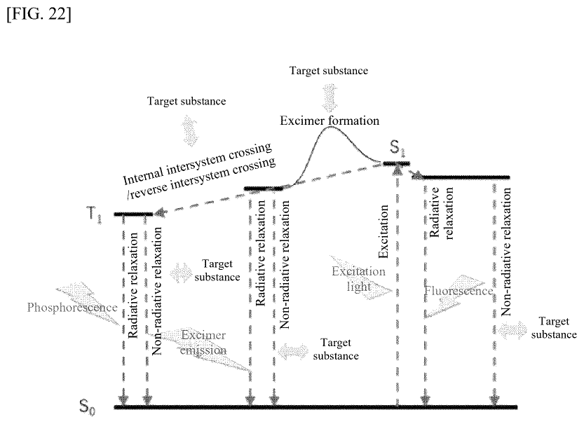

The scalable descriptor is constructed based on data acquired by a measurement modality using propagation of predetermined waves or a luminescent phenomenon. The predetermined waves include at least one of an acoustic wave and an electromagnetic wave (a microwave, a light wave, an X-ray, and the like). As an example of measurement means utilizing a luminescent phenomenon, that is, a light emission sensor, it is preferable to use at least one of an organic EL element (OLED) sensor including a reaction field having a microwell structure and a microwell sensor with a built-in OLED light source (see FIGS. 22 to 28 described later).

The measurement data, which is an element of the scalable descriptor, is acquired by a measurement modality having a scale corresponding to the characteristic and the like to be realized of the final product. The measurement scale is determined, for example, by selecting waves of appropriate frequency (wavelength) or wavenumber (wave duration or signal length), or by using a light emission sensor of appropriate wavelength. Specifically, for example, an ultrasound wave can be applied when a measurement scale is on the order of several tens of micrometers or more, a microwave or a millimeter wave can be applied when the measurement scale is on the order of several micrometers, and an X-ray or a light emission sensor can be applied when the measurement scale is on a smaller order. Furthermore, the selection of these modalities can be appropriately changed in consideration of the properties (frequency dependence of transmittance and reflectance, and the like) of the object, the surrounding environment, and the like.

In addition, the measurement data for constructing the scalable descriptor may include image information obtained from the object by using a predetermined wave, a light emission sensor, or the like. The image information may include an image of noise and/or distortion generated when the object is imaged. Furthermore, instead of or in addition to the image information itself, a feature amount extracted from the image information may be used. The feature amount means, for example, information extracted by performing statistical analysis such as frequency analysis or principal component analysis on the image information. Further, the image information may include a tomographic image. The tomographic image is obtained by imaging, on a two-dimensional or three-dimensional space, a change in intensity information or phase information regarding a reflected component or a transmitted component, which occurs due to structural or compositional discontinuity or non-uniformity in the object when a predetermined wave propagates in the object, together with the position information.

FIG. 3 is a schematic diagram conceptually illustrating a flow of data in the information processing apparatus 10 including the acquisition of measurement data regarding the object, the construction of a scalable descriptor from the data, the process control based on the descriptor, and the like. The acquirer 100 acquires first to third measurement data pieces as three types of data on different measurement scales regarding the structure and the like of the object. The descriptor construction section 110 constructs a scalable descriptor by the following procedure based on the acquired measurement data group.

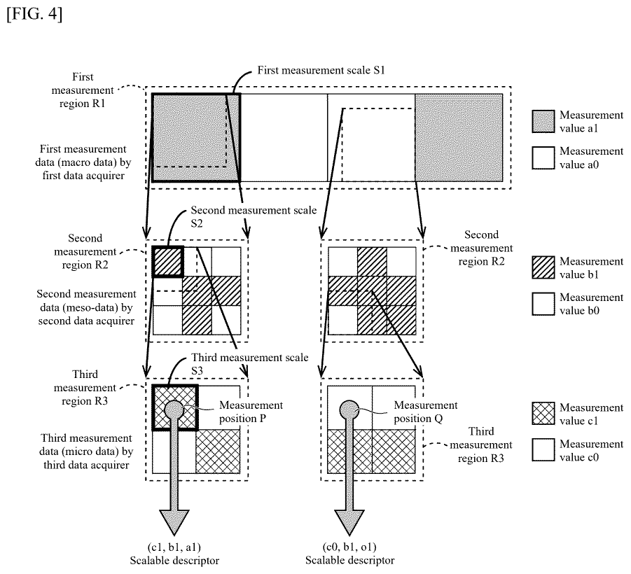

FIG. 4 is a schematic diagram illustrating a procedure of constructing a scalable descriptor from the measurement data group. In FIG. 4, the first data acquirer 101 acquires the first measurement data of a first measurement scale S1 in a first measurement region R1 as data of a large measurement scale (low measurement resolution) in the object by using, for example, a first measurement modality using long-wavelength waves. In the following description, it is based on the premise that the first measurement data can take a measurement value a0 or a measurement value a1 as two values (the same applies to the following measurement data). The first measurement modality is, for example, an ultrasound sensor using an ultrasound wave having a longer wavelength than the other measurement modalities. The first measurement data is, for example, a measurement value related to a layer structure of CFRP (carbon fiber composite material).

The second data acquirer 102 acquires the second measurement data (b0 or b1) of a second measurement scale S2 in a second measurement region R2 smaller than the first measurement region R1 by using a second measurement modality that uses a wave having a wavelength shorter than that of the wave used in the first measurement modality. The second measurement modality is, for example, an electromagnetic-wave sensor using an electromagnetic wave having a shorter wavelength than the first measurement modality. The second measurement data is, for example, a measurement value related to fiber dispersion or fiber orientation of the CFRP.

The third data acquirer 103 acquires the third measurement data (c0 or c1) of a third measurement scale S3 in a third measurement region R3 smaller than the second measurement region R2 by using a third measurement modality that uses a wave having a wavelength shorter than that of the wave used in the second measurement modality. The third measurement modality is, for example, a light emission sensor using a light wave or a terahertz wave having a wavelength shorter than that of the second measurement modality. The third measurement data is, for example, a measurement value related to a fiber-resin bonding state in the CFRP.

In FIG. 4, the second measurement region R2 is smaller than the first measurement region R1, and more preferably, has a size corresponding to the first measurement scale S1. The third measurement region R3 is smaller than the second measurement region R2, and more preferably, has a size corresponding to the second measurement scale S2. As described above, the first to third measurement regions R1 to R3 have a certain spatial relation in which one of the measurement regions is included in the other measurement region.

The descriptor construction section 110 connects the measurement values acquired by the first, second, and third measurement modalities at the same measurement position of the object to form a scalable descriptor. For example, in FIG. 4, the measurement values c1, b1, and a1 obtained at a measurement position P are respectively connected to the measurement values c0, b1, and a0 obtained at a measurement position Q. Then, the connected measurement value and data (for example, spatial coordinate information) indicating the measurement position (P, Q) are associated with each other to form a scalable descriptor. Note that in FIG. 4, each measurement modality acquires two values as the measurement values, but the configuration is not limited thereto. Each measurement modality may acquire multiple values equal to or greater than three values, or the measurement modalities may acquire different number of values.

As described in FIG. 4, it is desirable that the measurement values by the respective measurement modalities are such that the measurement positions can be coincided with each other or a relative positional relation can be grasped. In addition, it is desirable that the acquisition timings of the measurement values by the respective measurement modalities are the same or maintain a relatively constant temporal relation. For example, in a case where the position of the object changes over time (i.e., moves) in the design and manufacturing process, each acquisition timing is intentionally changed so as to be synchronized with the movement in position of the object so that the measurement values by the respective measurement modalities can be acquired from the same or equivalent object. Although the number of measurement modalities is three in FIG. 4, the number is not limited thereto, and may be two or four or more. As an example of the measurement modalities, as described above, it is assumed that an ultrasound sensor is used as the first measurement modality, an electromagnetic-wave sensor is used as the second measurement modality, and a light emission sensor is used as the third measurement modality. However, the measurement modalities are not limited thereto, and any other measurement modality may be adopted as long as the first to third measurement scales can be arranged in descending order.

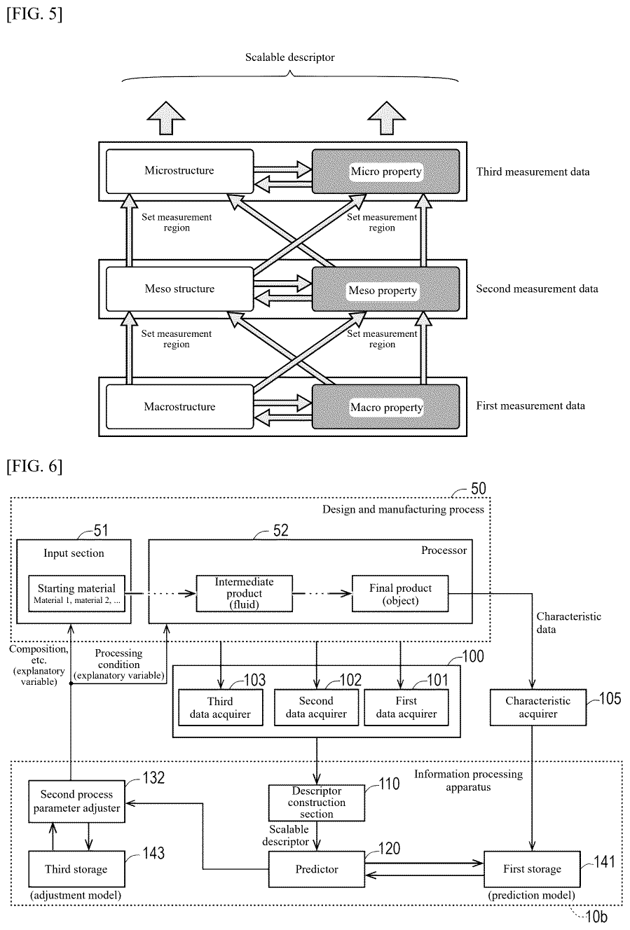

Next, the handling of the measurement scale and the data attribute of each piece of measurement data will be described with reference to FIG. 5. Measurement data acquired from an object is broadly divided into data related to the structure of the object (structural information) and data related to the property (property information). Which one of the structural information and the property information can be acquired or whether both of them can be acquired is determined by the measurement modality used for each data acquirer in the acquirer 100. Here, when all of the pieces of measurement data acquired by the respective data acquirers are related to the structural information, the descriptor construction section 110 constructs a scalable descriptor related to the structural information. Furthermore, if all are related to the property information, the descriptor construction section 110 constructs a scalable descriptor related to the property information. Furthermore, the measurement data acquired by each of the data acquirers may be a mixture of the structural information and the property information, or may include both the structural information and the property information as the measurement data of the same measurement scale. Preferably, the measurement data obtained by each of the data acquirers includes structural information and property information at as many measurement scales as possible.

### Second Embodiment

FIG. 6 is a diagram illustrating a configuration of a design and manufacturing process including an information processing apparatus 10b according to a second embodiment. As illustrated in FIG. 6, the information processing apparatus and the like according to the second embodiment are different from those of the first embodiment (FIG. 1) in that a second process parameter adjuster 132 is provided instead of the first process parameter adjuster 131, and a third storage 143 is provided instead of the second storage 142. On the other hand, the connection relation and operation of the other blocks are the same as those in FIG. 1. Therefore, the same contents as those in FIG. 1 are denoted by the same reference numerals and the description thereof will be omitted. Only differences from FIG. 1 are denoted by new reference numerals and the detail thereof will be described below.

In FIG. 6, the second process parameter adjuster 132 includes a third trained model, receives the characteristic and the like of a final product predicted by a predictor 120 using the third trained model, and outputs or adjusts a process parameter based on the input characteristic and the like.

The third storage 143 stores a plurality of sets of data, each of which includes characteristic data of the final product and a process parameter. This is training sample data (training data) and is used for training the third trained model used in the second process parameter adjuster 132. A training method for the third trained model will be described later.

### Third Embodiment

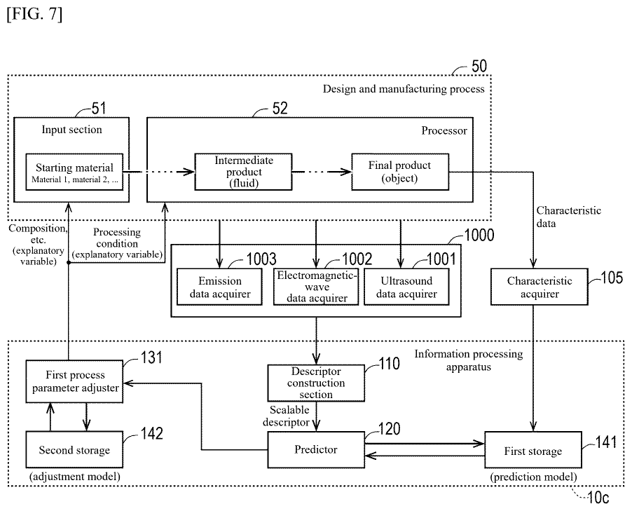

FIG. 7 is a diagram illustrating a configuration of a design and manufacturing process including an information processing apparatus 10c according to a third embodiment. As illustrated in FIG. 7, the information processing apparatus and the like according to the third embodiment are different from those in the first embodiment (FIG. 1) in the following points. The information processing apparatus and the like according to the third embodiment include an acquirer 1000 instead of the acquirer 100, and include an ultrasound data acquirer 1001, an electromagnetic-wave data acquirer 1002, and an emission data acquirer 1003 instead of the first data acquirer 101, the second data acquirer 102, and the third data acquirer 103, respectively. On the other hand, the connection relation and operation of the other blocks are the same as those in FIG. 1. Therefore, the same contents as those in FIG. 1 are denoted by the same reference numerals and the description thereof will be omitted. Only differences from FIG. 1 are denoted by new reference numerals and the detail thereof will be described below.

The ultrasound data acquirer 1001 includes, for example, an ultrasound probe (piezoelectric element or the like), and obtains a reception signal by irradiating an object with an ultrasound wave and receiving a transmitted wave and/or a reflected wave from the object. In a case where the object moves autonomously during acquisition of data over a wide area of the object, the ultrasound data acquirer 1001 applies ultrasound waves with the position and/or the direction of the ultrasound probe being fixed. In a case where the object stops during acquisition of data over a wide area of the object, the ultrasound data acquirer 1001 applies ultrasound waves with the position and/or the direction of the ultrasound probe being moved. If necessary, the ultrasound data acquirer 1001 generates an ultrasound image based on intensity information and phase information of the reception signal. The ultrasound image includes an ultrasound tomographic image representing tomographic information of the object, and may be a still image or a moving image. In addition, quantitative data such as a feature amount may be extracted from the ultrasound image.

The electromagnetic-wave data acquirer 1002 includes, for example, an antenna pattern including a metal waveguide or the like, an electrode, or the like on a dielectric substrate to spatially form a circuit including the object, and observes frequency characteristics including the resonance characteristics, the attenuation characteristics, and the like. Thus, the electromagnetic-wave data acquirer 1002 acquires data regarding the conductivity and dielectricity of the object. When acquiring data over a wide area of the object, the electromagnetic-wave data acquirer 1002 may acquire the data while moving the position and/or the direction of the antenna or the like.

The emission data acquirer 1003 acquires emission data as information corresponding to the state of the object by using a light-emitting material whose light emission behavior such as light emission intensity, light emission wavelength, and light emission lifetime changes according to the state (difference in type, change, or the like) of the object. More specifically, the emission data acquirer 1003 detects the behavior at a molecular level based on a change in the light emission spectrum caused by the intramolecular or intermolecular interaction of the light-emitting material generated according to the state of the object. By using the light-emitting material, the emission data acquirer 1003 can detect the state of the object with high accuracy even when there is no molecule that emits light by photoexcitation in the object. As the light-emitting material, for example, a luminescent DNA-type compound is used in which, in an artificial nucleic acid that forms an aggregate by recognizing a base sequence complementary to a natural nucleic acid, a nucleobase is partially substituted with a luminescent residue. Several thousands to several tens of thousands of kinds of luminescent DNA-type compounds are prepared according to the kinds of monomers and the sequences thereof, and emission spectrum data pieces of a wide variety of compounds with respect to the state of the object are simultaneously acquired, by which a slight difference or a complicated change in the state of the object is identified with high accuracy. Specific devices include a first sensing device and a second sensing device of the emission data acquirer to be described later (see FIGS. 22 to 28).

As described above, according to the first, second, and third embodiments, it is possible to acquire measurement data of different scales by a plurality of data acquirers included in the acquirer (100, 1000) and construct a multidimensional and multivariate scalable descriptor based on the measurement data. In consideration of recent improvement in computing machine performance and development of AI technology such as machine learning, image processing, and analysis technologies, application of these technologies to scalable descriptors is highly likely to enable extraction of not only information that can be easily recognized or understood by humans and can be verbalized and quantified, as acquired by conventional single-function sensors and the like, but also advanced information that cannot be currently understood or expressed by humans. For example, the third embodiment acquires structural information and viscoelastic information on the order of several tens of micrometers of the object using ultrasound waves, acquires electrical information on the order of micrometers of the object using electromagnetic waves, and acquires chemical information on the order of nanometers of the object using the light emission sensor. Thus, according to the third embodiment, it is possible to construct a scalable descriptor as a parameter capable of simultaneously expressing these pieces of information, and to simultaneously predict a plurality of characteristics and the like to be realized of a final product based on the scalable descriptor.

### (Processing of Predicting Characteristics and the Like and Processing of Adjusting Process Parameters)

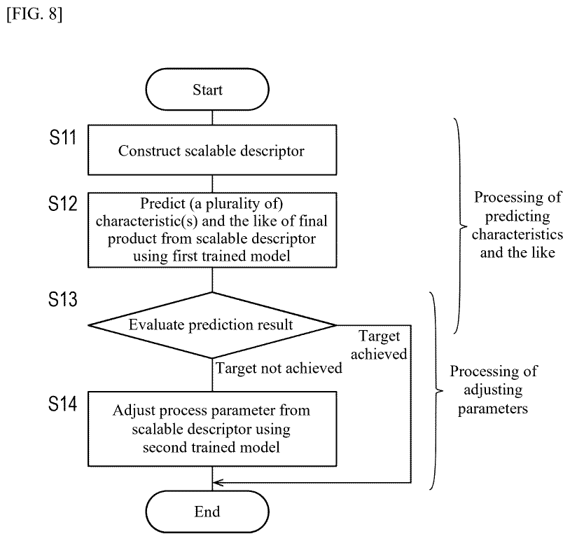

Next, processing of predicting characteristics and the like and processing of adjusting process parameters executed by the information processing apparatus 10 (FIG. 1) or 10c (FIG. 7) will be described with reference to FIGS. 8 to 10. FIG. 8 is a flowchart illustrating processing of predicting characteristics and the like and processing of adjusting parameters executed by the information processing apparatus 10 or 10c.

### (Step S11)

The acquirer 100 and the descriptor construction section 110 acquire measurement data from the object and construct a scalable descriptor. For example, the acquirer 100 acquires each piece of measurement data using the ultrasound sensor, the electromagnetic-wave sensor, and the light emission sensor. The descriptor construction section 110 constructs a scalable descriptor from each measurement data by predetermined processing.

### (Step S12)

The predictor 120 predicts the characteristic and the like of the final product from the scalable descriptor using the first trained model. Specifically, the scalable descriptor constructed in step S11 is input to the first trained model, and the characteristic and the like of the final product are predicted as an output relative to the input. The characteristic and the like are, for example, information or the like expressed as numerical data or a characteristic classification result. When the characteristic and the like are expressed as the classification result, the likelihood may be calculated for each classification candidate, and the classification with the highest likelihood may be obtained as the classification result.

### (Step S13)

The information processing apparatus 10 or 10c evaluates the prediction result (characteristic and the like of the final product) obtained in step S12. Specifically, the information processing apparatus 10 or 10c compares the characteristic and the like to be realized of the final product set in advance with the prediction result, and when they coincide with each other or the difference between them falls within a predetermined range (target achieved), the information processing apparatus 10 or 10c skips step S14 and ends the processing (end). On the other hand, when the difference between the characteristic and the like to be realized of the final product and the prediction result is not within the predetermined range (target not achieved), the information processing apparatus 10 or 10c advances the processing to step S14.

### (Step S14)

The first process parameter adjuster 131 adjusts the process parameter using the second trained model. Specifically, the scalable descriptor obtained in step S11 is input into the second trained model, and a process parameter is obtained as an output with respect to the input. The obtained process parameter may be displayed as a recommended adjustment value on a display or the like, or may be reflected in the composition and the like of the starting material in the input section 51 and the processing condition and the like used in the processor 52 as illustrated in FIGS. 1 and 7.

### (Training of First Trained Model)

FIG. 9 is a flowchart illustrating a process of a machine learning method for the first trained model. In the process illustrated in FIG. 9, the training sample data stored in the first storage 141 is used. The first storage 141 stores, as the training sample data, a plurality of sets of data, each of which is obtained by combining the scalable descriptor constructed by the descriptor construction section 110 and the characteristic data (ground truth data) of the final product acquired by the characteristic acquirer 105. In the following description, it is assumed that the information processing apparatus 10 or 10c functions as a learner to perform machine learning. However, the configuration is not limited thereto, and another cloud computer may perform machine learning as a learner (the same applies to processing of FIG. 10 described later). Furthermore, although a training method using a neural network formed by combining perceptrons in a learner (not illustrated) will be described below, the configuration is not limited thereto, and various methods can be adopted as long as they are supervised learning. For example, random forest, a support vector machine (SVM), boosting, a Bayesian network linear discriminant method, a non-linear discriminant method, or the like can be applied (the same applies to the processing in FIG. 10 described later).

### (Step S21)

The information processing apparatus 10 or 10c functioning as a learner reads training sample data as training data from the first storage 141. If this is the first time, the learner reads the first set of a scalable descriptor (hereinafter, also referred to as input data) of the object and the corresponding characteristic data (hereinafter, also referred to as output data) of a final product. Here, the scalable descriptor is constructed, for example, based on data acquired by the ultrasound sensor, the electromagnetic-wave sensor, and the light emission sensor.

### (Step S22)

The learner inputs, to the neural network, the input data (scalable descriptor) of the training sample data having been read. The neural network here is a network in which a weight designated by a user is set as an initial value if this is the first time.

### (Step S23)

The learner compares and evaluates the prediction result of the neural network, that is, the estimated characteristic and the like of the final product, with output data (characteristic data) that is ground truth data.

### (Step S24)

The learner adjusts the parameter (weight) of the neural network from the comparison result. The learner executes, for example, a back-propagation process to adjust and update the parameter so as to reduce an error in the comparison result.

### (Step S25)

When the process for all pieces of data of the training sample data has been completed (YES), the learner advances the processing to step S26. On the other hand, when the process for all pieces of data of the training sample data has not been completed (NO), the learner returns the processing to step S21 to read the next training sample data, and repeats the processes of step S21 and the subsequent steps.

### (Step S26)

The learner stores the first trained model constructed by the processes so far in a storage area (for example, a memory in the predictor 120) and ends the processing (end). Thereafter, the process of FIG. 8 (step S12) is performed using the first trained model.

### (Training of Second Trained Model)

FIG. 10 is a flowchart illustrating a process of the machine learning method for the second trained model. In the processing illustrated in FIG. 10, the training sample data stored in the second storage 142 is used. The second storage 142 stores, as the training sample data, a plurality of sets of data, each of which is obtained by combining the scalable descriptor and the process parameter (ground truth data).

### (Step S31)

The information processing apparatus 10 or 10c functioning as a learner reads the training sample data as training data from the second storage 142. If this is the first time, the learner reads the first set of a scalable descriptor (hereinafter, also referred to as input data) of the object and the data (hereinafter, also referred to as output data) of a corresponding process parameter.

### (Step S32)

The learner inputs, to the neural network, the input data (scalable descriptor) of the training sample data having been read. The neural network here is a network in which a weight designated by the user is set as an initial value if this is the first time.

### (Step S33)

The learner compares and evaluates the prediction result of the neural network, that is, the estimated process parameter data, with output data (characteristic data) that is ground truth data.

### (Step S34)

The learner adjusts the parameter (weight) of the neural network from the comparison result. The learner executes, for example, a back-propagation process to adjust and update the parameter so as to reduce an error in the comparison result.

### (Step S35)

When the process for all pieces of data of the training sample data has been completed (YES), the learner advances the processing to step S36. On the other hand, when the process for all pieces of data of the training sample data has not been completed (NO), the learner returns the processing to step S31 to read the next training sample data, and repeats the processes of step S31 and the subsequent steps.

### (Step S36)

The learner stores the second trained model constructed by the processes so far in a storage area (for example, a memory in the first process parameter adjuster 131) and ends the processing (end). Thereafter, the process of FIG. 8 (step S14) is performed using the second trained model.

In FIG. 10, the learner may reverse the relation between the input data and the output data. In this case, the learner is configured to predict the scalable descriptor from the process parameter using the process parameter as input data and the scalable descriptor as output data, and to compare and evaluate the result with ground truth data (scalable descriptor).

### (Processing of Predicting Characteristics and the Like and Processing of Adjusting Process Parameters)

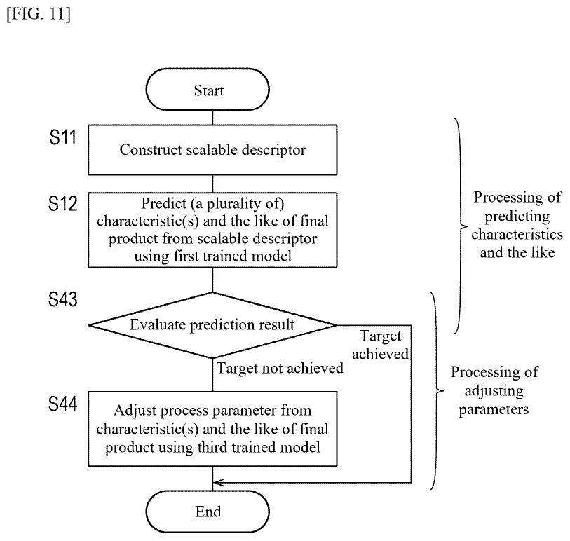

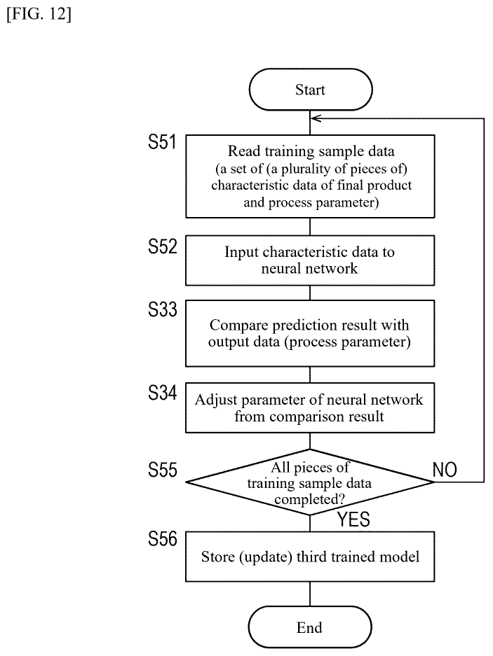

Next, processing of predicting characteristics and the like and processing of adjusting process parameters executed by the information processing apparatus 10b (FIG. 6) will be described with reference to FIGS. 11 and 12. FIG. 11 is a flowchart illustrating the processing of predicting characteristics and the like and processing of adjusting parameters executed by the information processing apparatus 10b. The flowchart in FIG. 11 is obtained by changing the content of some of steps in FIG. 8. Therefore, the same steps as those in FIG. 8 are denoted by the same reference numerals and the description thereof will be omitted. Only differences from FIG. 8 are denoted by new reference numerals and the details thereof will be described below.

### (Step S43)

The information processing apparatus 10b evaluates the prediction result (characteristic and the like of the final product) obtained in step S12. Specifically, the information processing apparatus 10b compares the characteristic and the like to be realized of the final product set in advance with the prediction result, and when they coincide with each other or the difference between them falls within a predetermined range (target achieved), the information processing apparatus 10b skips step S44 and ends the processing (end). On the other hand, when the difference between the characteristic and the like to be realized of the final product and the prediction result is not within the predetermined range (target not achieved), the information processing apparatus 10b advances the processing to step S44.

### (Step S44)

The second process parameter adjuster 132 adjusts the process parameter using the third trained model. Specifically, the result of predicting the characteristic and the like of the final product obtained in step S12 is input to the third trained model, and the process parameter is obtained as an output with respect to the input. The obtained process parameter may be displayed as a recommended adjustment value on a display or the like, or may be reflected in the composition and the like of the starting material in the input section 51 and the processing condition and the like used in the processor 52 as illustrated in FIG. 6.

### (Training of Third Trained Model)

FIG. 12 is a flowchart illustrating a process of the machine learning method for the third trained model. In the processing illustrated in FIG. 12, the training sample data stored in the third storage 143 is used. The third storage 143 stores, as the training sample data, a plurality of sets of data, each of which is obtained by combining the characteristic and the like of the final product and a process parameter (ground truth data). The flowchart in FIG. 12 is obtained by changing the content of some of steps in FIG. 10. Therefore, the same steps as those in FIG. 10 are denoted by the same reference numerals and the description thereof will be omitted. Only differences from FIG. 10 are denoted by new reference numerals and the details thereof will be described below.

### (Step S51)

The information processing apparatus 10b functioning as a learner reads the training sample data as training data from the third storage 143. If this is the first time, the learner reads the first set of the characteristic data (hereinafter, also referred to as input data) of the final product and the data (hereinafter, also referred to as output data) of a corresponding process parameter.

### (Step S52)

The learner inputs, to the neural network, the input data (characteristic data) of the training sample data having been read. The neural network here is a network in which a weight designated by the user is set as an initial value if this is the first time.

### (Step S55)

When the process for all pieces of data of the training sample data has been completed (YES), the learner advances the processing to step S56. When the process has not been completed (NO), the learner returns the processing to step S51 to read the next training sample, and repeats the processes of step S51 and the subsequent steps.

### (Step S56)

The learner stores the third trained model constructed by the processes so far in a storage area (for example, a memory in the second process parameter adjuster 132) and ends the processing (end). Thereafter, the process of FIG. 11 (step S44) is performed using the third trained model.

In FIG. 12, the learner may reverse the relation between the input data and the output data. In this case, the learner is configured to predict the characteristic data of the final product from the process parameter using the process parameter as input data and the characteristic data of the final product as output data, and to compare and evaluate the result with ground truth data (characteristic data).

### (Processing of Predicting Characteristics and the Like and Processing of Adjusting Process Parameters)

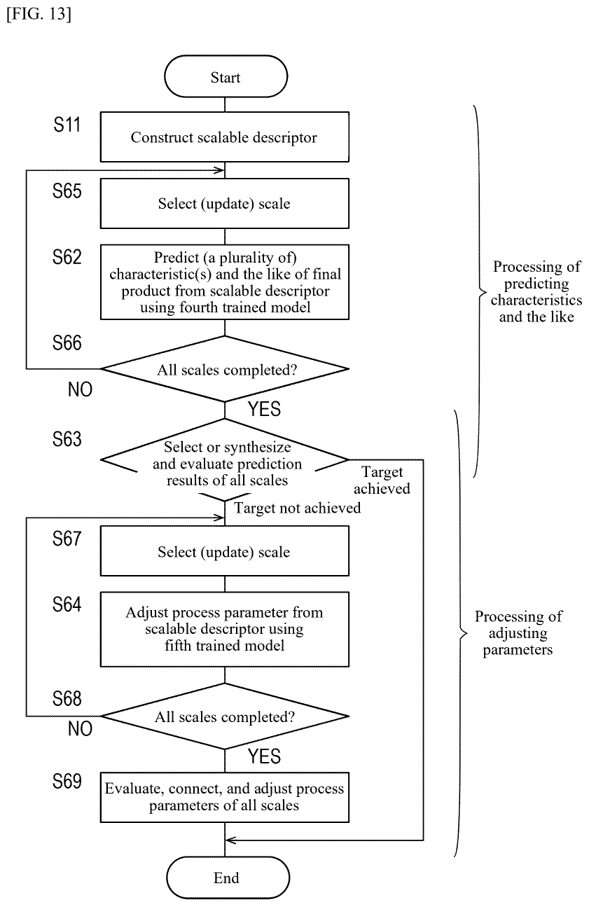

Next, another example of the processing of predicting characteristics and the like and processing of adjusting process parameters executed by the information processing apparatus 10 (FIG. 1) or 10c (FIG. 7) will be described with reference to FIGS. 13 to 15. FIG. 13 is a flowchart illustrating the processing of predicting characteristics and the like and processing of adjusting parameters executed by the information processing apparatus 10 or 10c. The flowchart in FIG. 13 is obtained by changing the content of some of steps in FIG. 8 and adding some steps. Therefore, the same steps as those in FIG. 8 are denoted by the same reference numerals and the description thereof will be omitted. Only differences from FIG. 8 are denoted by new reference numerals and the details thereof will be described below.

### (Step S65)

The information processing apparatus 10 or 10c selects one of all the scales of the measurement data acquired by the acquirer 100.

### (Step S62)

The predictor 120 predicts the characteristics and the like of the final product from the scalable descriptor corresponding to the scale by using the fourth trained model corresponding to the selected scale. Specifically, the scalable descriptor corresponding to the selected scale among the scalable descriptors constructed in step S11 is input to the fourth trained model corresponding to the scale, and the characteristic and the like of the final product are predicted as an output with respect to the input.

### (Step S66)

When the process is completed for all scales (YES), the information processing apparatus 10 or 10c advances the processing to S63. On the other hand, when the process is not completed (NO), the information processing apparatus 10 or the like returns the processing to step S65 to select the next scale, and repeats the processes of step S65 and the subsequent steps.

### (Step S63)

The information processing apparatus 10 or 10c selects a prediction result (the characteristic and the like of the final product) from the prediction results regarding all the scales obtained in step S62 based on a prescribed criterion or synthesizes the prediction results by a prescribed method and then evaluates them. Specifically, the information processing apparatus 10 or 10c compares the characteristic and the like to be realized of the final product set in advance with the prediction result, and when they coincide with each other or the difference between them falls within a predetermined range (target achieved), the information processing apparatus 10 or 10c skips steps S67 to S69 and ends the processing (end). On the other hand, when the difference between the characteristic and the like to be realized of the final product and the prediction result is not within the predetermined range (target not achieved), the information processing apparatus 10 or 10c advances the processing to step S67. Further, the selection based on the prescribed criterion means, for example, selecting the prediction result closest to or farthest from the characteristic and the like to be realized among the prediction results for all scales. The synthesis by a prescribed method means, for example, performing weighted average on or averaging the prediction results of all the scales or selected some of the scales.

### (Step S67)

The information processing apparatus 10 or 10c selects one of all the scales of the measurement data acquired by the acquirer 100.

### (Step S64)

The first process parameter adjuster 131 outputs a process parameter from the scalable descriptor corresponding to the selected scale, using the fifth trained model corresponding to the selected scale. Specifically, the scalable descriptor corresponding to the selected scale among the scalable descriptors constructed in step S11 is input to the fifth trained model corresponding to the selected scale, and the process parameter is obtained as an output with respect to the input.

### (Step S68)

When the process has been completed for all the scales (YES), the information processing apparatus 10 or 10c advances the processing to step S69. When the process has not been completed (NO), the information processing apparatus 10 or 10c returns the processing to step S67 to read the next scale and repeats the processes of step S67 and the subsequent steps.

### (Step S69)

The information processing apparatus 10 or 10c selects a process parameter from the process parameters related to all the scales obtained in step S64 based on a prescribed criterion or synthesizes the process parameters by a prescribed method and outputs them. Here, the selection based on the predetermined criterion means, for example, selecting the process parameter related to the scale corresponding to the prediction result determined to be closest to or farthest from the characteristic and the like to be realized in step S63. The synthesis by a prescribed method means, for example, performing weighted average on or averaging the process parameters related to all the scales or selected some of the scales. The obtained process parameter may be displayed as a recommended adjustment value on a display or the like, or may be reflected in the composition and the like of the starting material in the input section 51 and the processing condition and the like used in the processor 52 as illustrated in FIGS. 1 and 7.

### (Training of Fourth Trained Model)

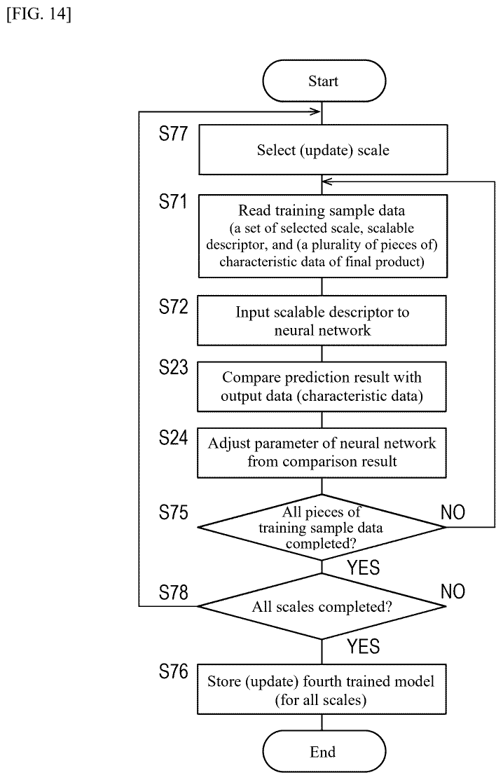

FIG. 14 is a flowchart illustrating a process of the machine learning method for the fourth trained model. In the processing illustrated in FIG. 14, the training sample data stored in the first storage 141 is used. The first storage 141 stores, as the training sample data, a plurality of sets of data, each of which is obtained by combining the scalable descriptor (including information about the scale) constructed by the descriptor construction section 110 and the characteristic data (ground truth data) of the final product acquired by the characteristic acquirer 105. The flowchart in FIG. 14 is obtained by changing the content of some of steps in FIG. 9 and adding some steps. Therefore, the same steps as those in FIG. 9 are denoted by the same reference numerals and the description thereof will be omitted. Only differences from FIG. 9 are denoted by new reference numerals and the details thereof will be described below.

### (Step S77)

The information processing apparatus 10 or 10c functioning as a learner selects one of all the scales of the measurement data acquired by the acquirer 100.

### (Step S71)

The learner reads, as the training data, the selected scale and training sample data corresponding to the scale from the first storage 141. If this is the first time, the learner reads the first set of a scalable descriptor (hereinafter, also referred to as input data) corresponding to the selected scale of the object and the corresponding characteristic data (hereinafter, also referred to as output data) of a final product.

### (Step S72)

The learner inputs, to the neural network, the input data (selected scale and the scalable descriptor corresponding to the scale) of the training sample data having been read. The neural network here is a network in which a weight designated by the user is set as an initial value if this is the first time.

### (Step S75)

When the process for all pieces of data of the training sample data has been completed (YES), the learner advances the processing to step S78. On the other hand, when the process for all pieces of data of the training sample data has not been completed (NO), the learner returns the processing to step S71 to read the next training sample data, and repeats the processes of step S71 and the subsequent steps.

### (Step S78)

When the process has been completed for all scales (YES), the learner advances the processing to step S76. When the process has not been completed (NO), the learner returns the processing to step S77 to select the next scale, and repeats the processes of step S77 and the subsequent steps.

### (Step S76)

The learner stores the fourth trained model constructed by the processes so far in a storage area (for example, a memory in the predictor 120) and ends the processing (end). Thereafter, the process of FIG. 13 (step S62) is performed using the fourth trained model.

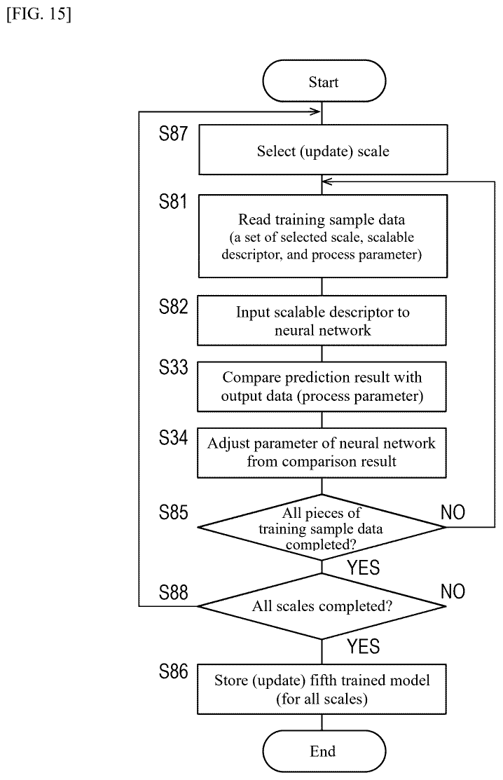

In FIG. 14, the scale is selected (step S77), and then, the training sample data corresponding to the scale is read (step S71). However, the configuration is not limited thereto, and a procedure may be adopted in which the training sample data is read and then a scale is selected (the same applies to FIG. 15 described later).

### (Training of Fifth Trained Model)

FIG. 15 is a flowchart illustrating a process of the machine learning method for the fifth trained model. In the processing illustrated in FIG. 15, the training sample data stored in the second storage 142 is used. The second storage 142 stores, as the training sample data, a plurality of sets of data, each of which is obtained by combining the scalable descriptor (including information about the scale) and the process parameter (ground truth data). The flowchart in FIG. 15 is obtained by changing the content of some of steps in FIG. 10 and adding some steps. Therefore, the same steps as those in FIG. 10 are denoted by the same reference numerals and the description thereof will be omitted. Only differences from FIG. 10 are denoted by new reference numerals and the details thereof will be described below.

### (Step S87)

The information processing apparatus 10 or 10c functioning as a learner selects one of all the scales of the measurement data acquired by the acquirer 100.

### (Step S81)

The learner reads, as the training data, the selected scale and training sample data corresponding to the scale from the second storage 142. If this is the first time, the learner reads the first set of a scalable descriptor (hereinafter, also referred to as input data) corresponding to the selected scale of the object and the data (hereinafter, also referred to as output data) of a corresponding process parameter.

### (Step S82)

The learner inputs, to the neural network, the input data (selected scale and the scalable descriptor corresponding to the scale) of the training sample data having been read. The neural network here is a network in which a weight designated by the user is set as an initial value if this is the first time.

### (Step S85)

When the process for all pieces of data of the training sample data has been completed (YES), the learner advances the processing to step S88. On the other hand, when the process for all pieces of data of the training sample data has not been completed (NO), the learner returns the processing to step S81 to read the next training sample data, and repeats the processes of step S81 and the subsequent steps.

### (Step S88)

When the process has been completed for all scales (YES), the learner advances the processing to step S86. When the process has not been completed (NO), the learner returns the processing to step S87 to select the next scale, and repeats the processes of step S87 and the subsequent steps.

### (Step S86)

The learner stores the fifth trained model constructed by the processes so far in a storage area (for example, a memory in the first process parameter adjuster 131) and ends the processing (end). Thereafter, the processing of FIG. 13 (step S64) is performed using the fifth trained model.

In FIG. 15, the learner may reverse the relation between the input data and the output data. In this case, the learner is configured to predict the scalable descriptor from the process parameter using the process parameter as input data and the scalable descriptor as output data, and to compare and evaluate the result with ground truth data (scalable descriptor).

As described above, the information processing apparatus 10, 10b, or 10c according to the present embodiment is configured to, in a design and manufacturing process for obtaining a final product having predetermined characteristics and the like from a starting material through an intermediate product, acquire measurement data indicating the structures and/or properties of the final product, intermediate product, and/or starting material (i.e., the structure and the like of the object), and construct a multidimensional and multivariate scalable descriptor, thereby being capable of simultaneously predicting and evaluating a plurality of characteristics and the like of the final product and appropriately adjusting a process parameter based on the result.

### EXAMPLES

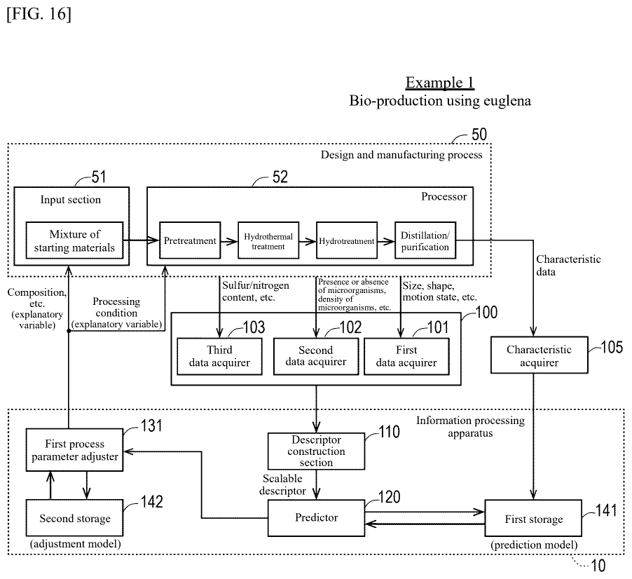

Application examples of the information processing apparatus and the like according to the present embodiment will be described below with reference to FIGS. 16 to 21.

### Example 1

FIG. 16 is a diagram illustrating a schematic configuration of a design and manufacturing process in Example 1. Example 1 shows an example in which the acquirer 100, the characteristic acquirer 105, and the information processing apparatus 10 are applied to the applicable type of industry 1 (bio-production using Euglena). Note that the information processing apparatus 10b or 10c may be applied instead of the information processing apparatus 10 (the same applies to Examples 2 to 6 below). The acquirer 100 includes first to third data acquirers 101 to 103. The acquirer 100 acquires measurement data on different measurement scales from a starting material, an intermediate product (such as a culture solution), and/or a final product during a mixing process, a pretreatment, a hydrothermal treatment, a hydrotreatment, and a distillation/purification treatment in the input section 51 and the processor 52. In the example illustrated in FIG. 16, the first data acquirer 101 acquires the size, shape, motion state, and the like of Euglena or the like as measurement data with a large measurement scale, the second data acquirer 102 acquires the presence or absence, density, and the like of microorganisms as measurement data with a smaller measurement scale, and the third data acquirer 103 acquires a sulfur content, a nitrogen content, and the like as measurement data with a still smaller measurement scale. The descriptor construction section 110 constructs scalable descriptors from these pieces of measurement data. The predictor 120 predicts the characteristics and the like of the final product using all or some of the obtained scalable descriptors. Further, the first process parameter adjuster 131 outputs appropriate values of the process parameters for the input section 51 and the processor 52 based on the scalable descriptors, and adjusts the process parameters based on the appropriate values. Note that the parameters acquired by the first to third data acquirers 101 to 103 are not limited to those described above, and may be any other parameters as long as the parameters are arranged in the descending order of measurement scale (the same applies to Examples 2 to 6 below).

### Example 2

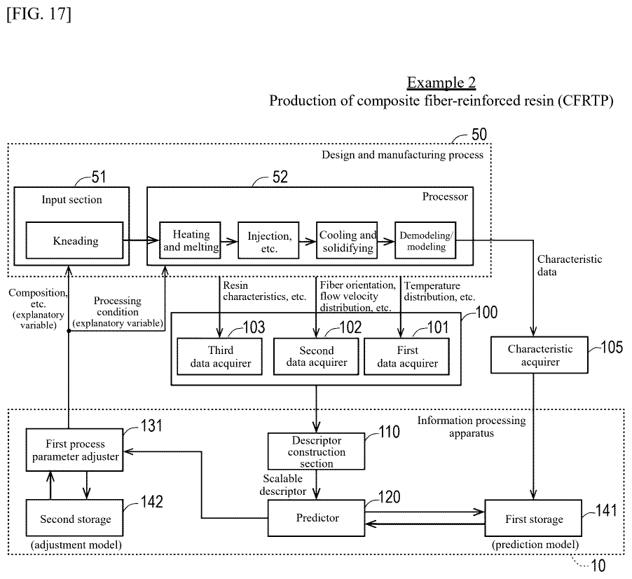

FIG. 17 is a diagram illustrating a schematic configuration of a design and manufacturing process in Example 2. Example 2 shows an example in which the acquirer 100, the characteristic acquirer 105, and the information processing apparatus 10 are applied to the applicable type of industry 2 (production of composite materials). The acquirer 100 includes first to third data acquirers 101 to 103. The acquirer 100 acquires measurement data on different measurement scales from a starting material, an intermediate product (e.g., resin in a molten state), and/or a final product during a kneading process, a heating and melting process, an injection (rolling and casting) process, a cooling and solidifying process, and a demolding/molding process performed by the input section 51 and the processor 52. In the example illustrated in FIG. 17, the first data acquirer 101 acquires a temperature distribution and the like as measurement data with a large measurement scale. The second data acquirer 102 acquires, as measurement data with a smaller measurement scale, fiber orientation, a flow velocity distribution of molten resin, and the like. The third data acquirer 103 acquires resin characteristics (dielectric constant and the like) and the like as measurement data with a still smaller measurement scale. The descriptor construction section 110 constructs scalable descriptors from these pieces of measurement data. The predictor 120 predicts the characteristics and the like of the final product using all or some of the obtained scalable descriptors. Further, the first process parameter adjuster 131 outputs appropriate values of the process parameters for the input section 51 and the processor 52 based on the scalable descriptors, and adjusts the process parameters based on the appropriate values.

### Example 3

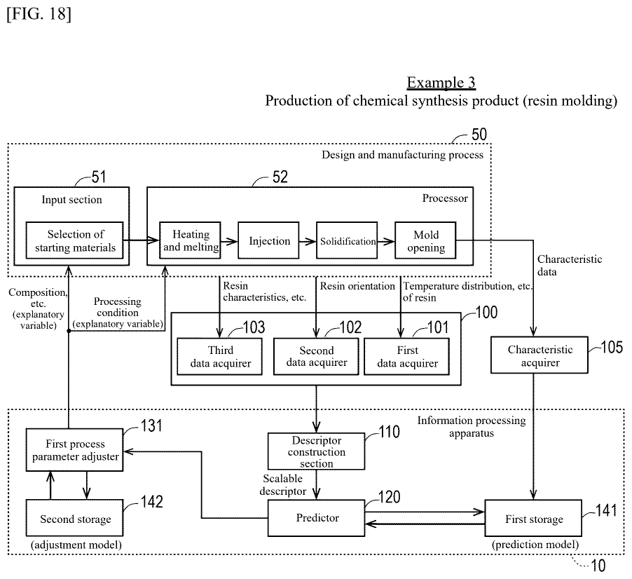

FIG. 18 is a diagram illustrating a schematic configuration of a design and manufacturing process in Example 3. Example 3 shows an example in which the acquirer 100, the characteristic acquirer 105, and the information processing apparatus 10 are applied to, in particular, a resin molding field as the applicable type of industry 3 (production of chemical synthesis products). The acquirer 100 includes first to third data acquirers 101 to 103. The acquirer acquires measurement data on different measurement scales from a starting material, an intermediate product (such as fluid resin), and/or a final product during a selection process, a heating and melting process, an injection process, a solidification process (involving pressure keeping and cooling), and mold opening by the input section 51 and the processor 52. In the example illustrated in FIG. 18, the first data acquirer 101 acquires a temperature distribution, a flow velocity distribution, and the like of a resin or the like as measurement data with a large measurement scale. The second data acquirer 102 acquires the resin orientation and the like as measurement data with a smaller measurement scale. The third data acquirer 103 acquires resin characteristics (dielectric constant and the like) and the like as measurement data with a still smaller measurement scale. The descriptor construction section 110 constructs scalable descriptors from these pieces of measurement data. The predictor 120 predicts the characteristics and the like of the final product using all or some of the obtained scalable descriptors. Further, the first process parameter adjuster 131 outputs appropriate values of the process parameters for the input section 51 and the processor 52 based on the scalable descriptors, and adjusts the process parameters based on the appropriate values.

### Example 4

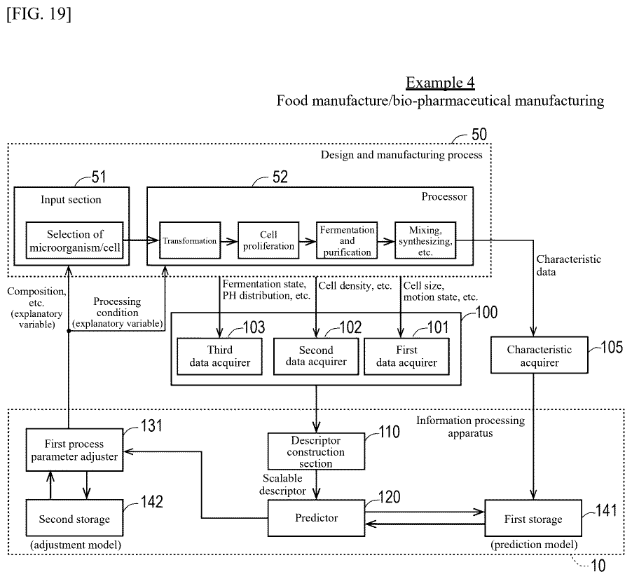

FIG. 19 is a diagram illustrating a schematic configuration of a design and manufacturing process in Example 4. Example 4 shows an example in which the acquirer 100, the characteristic acquirer 105, and the information processing apparatus 10 are applied to the applicable type of industry 4 (food manufacture) or applicable type of industry 6 (bio-pharmaceutical manufacturing). The acquirer 100 includes first to third data acquirers 101 to 103, and acquires measurement data on different measurement scales from a starting material, an intermediate product (culture solution and the like), and/or a final product during a selection process, a transformation process, a cell growth process, a fermentation/purification process, and a mixing/synthesis process by the input section 51 and the processor 52. In the example illustrated in FIG. 19, the first data acquirer 101 acquires a cell size, a motion state, and the like as measurement data with a large measurement scale. The second data acquirer 102 acquires the cell density and the like as the measurement data with a smaller measurement scale. The third data acquirer 103 acquires the fermentation state, the PH distribution, and the like as measurement data with a still smaller measurement scale. The descriptor construction section 110 constructs scalable descriptors from these pieces of measurement data. The predictor 120 predicts the characteristics and the like of the final product using all or some of the obtained scalable descriptors. Further, the first process parameter adjuster 131 outputs appropriate values of the process parameters for the input section 51 and the processor 52 based on the scalable descriptors, and adjusts the process parameters based on the appropriate values.

### Example 5

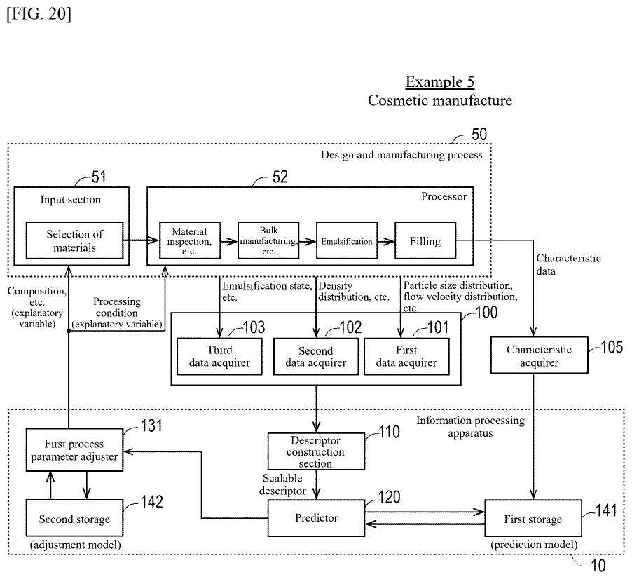

FIG. 20 is a diagram illustrating a schematic configuration of a design and manufacturing process in Example 5. Example 5 shows an example in which the acquirer 100, the characteristic acquirer 105, and the information processing apparatus 10 are applied to the applicable type of industry 5 (cosmetic manufacture). The acquirer 100 includes first to third data acquirers 101 to 103. The acquirer 100 acquires measurement data on different measurement scales from a starting material, an intermediate product (fluid), and/or a final product during a selection process, a material inspection, a bulk manufacturing (mixing) process, an emulsification process, and a filling process by the input section 51 and the processor 52. In the example illustrated in FIG. 20, the first data acquirer 101 acquires a particle size distribution, a flow velocity distribution, and the like as measurement data with a large measurement scale. The second data acquirer 102 acquires a density distribution and the like as measurement data with a smaller measurement scale. The third data acquirer 103 acquires the emulsification state and the like as measurement data with a still smaller measurement scale. The descriptor construction section 110 constructs scalable descriptors from these pieces of measurement data. The predictor 120 predicts the characteristics and the like of the final product using all or some of the obtained scalable descriptors. Further, the first process parameter adjuster 131 outputs appropriate values of the process parameters for the input section 51 and the processor 52 based on the scalable descriptors, and adjusts the process parameters based on the appropriate values.

### Example 6

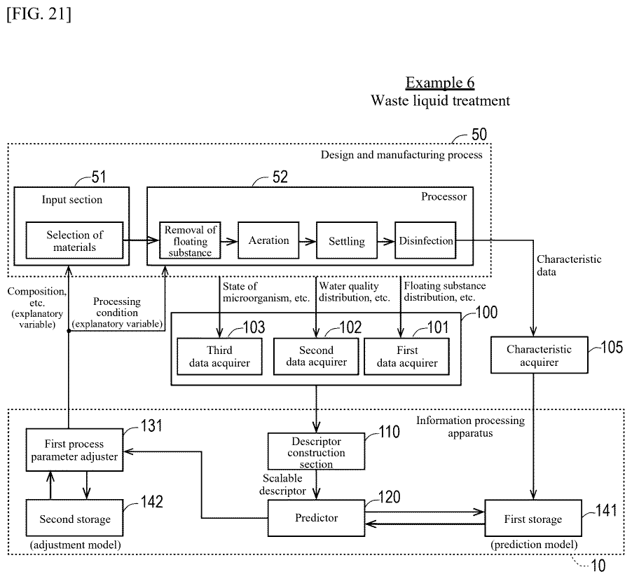

FIG. 21 is a diagram illustrating a schematic configuration of a design and manufacturing process in Example 6. Example 6 shows an example in which the acquirer 100, the characteristic acquirer 105, and the information processing apparatus 10 are applied to the applicable type of industry 7 (waste liquid treatment). The acquirer 100 includes first to third data acquirers 101 to 103. The acquirer 100 acquires measurement data on different measurement scales from an input matter, an intermediate product (such as sewage), and/or a final output matter during a selection process, a floating substance removal process, an aeration process, a settling process, and a disinfectant treatment by the input section 51 and the processor 52. In the example illustrated in FIG. 21, the first data acquirer 101 acquires a floating substance distribution and the like as measurement data with a large measurement scale. The second data acquirer 102 acquires water quality distribution and the like as measurement data with a smaller measurement scale. The third data acquirer 103 acquires the state of microorganism and the like as measurement data with a still smaller measurement scale. The descriptor construction section 110 constructs scalable descriptors from these pieces of measurement data. The predictor 120 predicts the characteristics and the like of the final output matter using all or some of the scalable descriptors. Further, the first process parameter adjuster 131 outputs appropriate values of the process parameters for the input section 51 and the processor 52 based on the scalable descriptors, and adjusts the process parameters based on the appropriate values.

As described above, the information processing apparatus and the like according to the present embodiment can be applied to various types of industry, and can simultaneously predict a plurality of characteristics of a final product in each applicable type of industry using a scalable descriptor constructed from measurement data to efficiently perform manufacturing.

With reference to FIGS. 22 to 28, first and second sensing devices will be described below as examples of the light emission sensor of the emission data acquirer 1003.

### (First Sensing Device of Emission Data Acquirer 1003)

The first sensing device uses microwells capable of forming a plurality of reaction fields on a plane. Different light-emitting materials are prepared on the wells in advance, and a sampled object is injected to acquire emission spectrum data. Then, the state of the object is represented as data from a plurality of pieces of emission spectrum data.

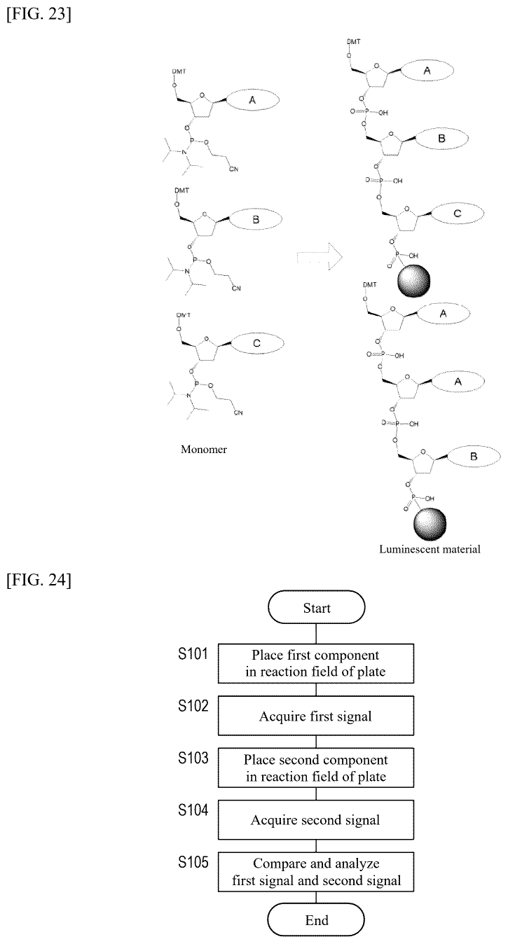

In the following, a plate in which a plurality of wells is regularly arranged is used. In the plate having such wells, the wells (reaction fields) are physically separated from each other by partition walls. Thus, target substances and luminescent dye molecules in the adjacent reaction fields are less likely to be mixed, whereby accurate analysis can be easily performed. FIG. 22 is a diagram for describing luminescent dye molecules in the first sensing device of the emission data acquirer 1003. FIG. 23 is a diagram for describing a production dye molecule of a luminescent dye molecule. FIG. 24 is a flowchart illustrating an analysis method using luminescent dye molecules.

### 1. Luminescent Dye Molecule (Light-Emitting Material)

The luminescent dye molecule of the present embodiment is a molecule having a main chain having one or more structural units, each of which contains a sugar structure derived from pentose or hexose and a phosphoester bond bonded to the sugar structure, and one or more chromophores or luminophores bonded to the sugar structure.

Furthermore, the luminescent dye molecule of the present embodiment emits, with respect to single excitation light, two or more kinds of light selected from the group consisting of fluorescence, phosphorescence, excimer emission, exciplex emission, thermally activated delayed fluorescence, excited-state intramolecular proton emission, triplet-triplet annihilation emission, twisted intramolecular charge transfer emission, and aggregation-induced emission.

The luminescent dye molecule of the present embodiment can be used for analysis of the structure, state, and the like of a specific target substance. Specifically, when the luminescent dye molecule and the target substance are allowed to interact with each other, the structure and the electronic state of the chromophore or the luminophore in the luminescent dye molecule change, and thus a complicated light emission behavior different from that in the case of the luminescent dye molecule alone is obtained. For example, as illustrated in FIG. 22, when a luminescent dye molecule that emits three different types of light, i.e., fluorescence, phosphorescence, and excimer emission, in response to single excitation light interacts with a target substance, the processes of generating the fluorescence, the phosphorescence, and the excimer emission change due to the interaction between the target substance and the luminescent dye molecule, and the wavelengths and lifetimes of the respective types of light change. Therefore, a large amount of complicated data in which the above-described types of light are combined is obtained according to the structure, the state, and the like of the target substance. According to such a complicated and large amount of data, it is possible to grasp the structure, the state, and the like of the target substance in very detail.

A specific structure of the luminescent dye molecule will be described below.

It is sufficient that the main chain of the luminescent dye molecule has one or more structural units, each of which includes a sugar structure derived from pentose or hexose and a phosphoester bond bonded to the sugar structure. The main chain may include only one of the structural units, or may include a plurality of the structural units. That is, the main chain may have a structure having the sugar structure and a phosphoester bond bonded to the sugar structure, or may have the sugar structures and the phosphoester bonds in an alternating manner. Usually, both ends of the main chain of the luminescent dye molecule have a sugar structure, so that the main chain has one more sugar structures than the phosphoester bonds. When the main chain includes a plurality of structural units, the plurality of structural units may be the same as or different from each other.

The number of the structural units included in the main chain of the luminescent dye molecule is appropriately selected depending on the type of the target substance or the like, and is preferably two or more and six or less. When the number of the structural units increases, the luminescent dye molecule tends to act specifically on the target substance. However, in the present embodiment, it is preferable to obtain a large amount of data by allowing the luminescent dye molecule to interact with the target substance at various positions. Therefore, it is preferable that the luminescent dye molecule and the target substance have moderate (not excessive) specificity, and the number of the structural units is preferably six or less.

Note that the main chain of the luminescent dye molecule may partially include a structure other than the pentose- or hexose-derived sugar structure and the structural unit containing a phosphoester bond, as long as the object and the effects of the present embodiment are not impaired. In addition, the structures of both ends of the main chain are not particularly limited, and for example, can be various structures such as an OH group and an alkoxy group.

Here, examples of the pentose include ribose, deoxyribose, and xylose. On the other hand, specific examples of the hexose include allose, glucose, and mannose. Particularly, when the sugar structure is derived from ribose or deoxyribose, the main chain of the luminescent dye molecule has the same structure as the main chain of DNA or RNA and thus tends to interact with DNA or RNA. Therefore, this configuration is preferable.

When the structural unit contains a structure derived from ribose or deoxyribose, the phosphoester bond is preferably bonded to the carbon at position 3 and the carbon at position 5 of ribose or deoxyribose. Furthermore, the chromophore or luminophore described below is preferably bonded to the position 1 of ribose or deoxyribose. That is, the luminescent dye molecule of the present embodiment preferably includes a structure represented by the following general formula (1a) or (1b).

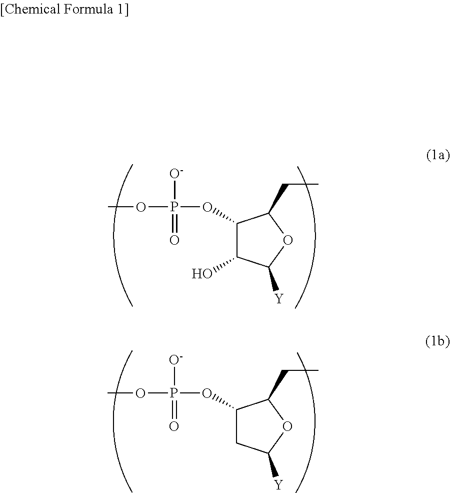

In the general formulae (1a) and (1b), Y represents a chromophore or a luminophore which will be described later.

On the other hand, the chromophore or the luminophore included in the luminescent dye molecule may have a structure in which a single chromophore or luminophore emits a predetermined type of light in response to single excitation light, or a plurality of chromophores or luminophores act to emit predetermined light. The chromophore or the luminophore is preferably bonded to the sugar structure of the main chain so that the sugar structure is in a β-form. Here, the term “chromophore” refers to a structure that absorbs light having a wavelength of 300 nm or more, and the term “luminophore” refers to a structure that absorbs light having a wavelength of 300 nm or more and emits light.

The number of the chromophore or the luminophore included in the luminescent dye molecule may be only one as long as the luminescent dye molecule can emit a plurality of types of luminescence. However, in order to make the luminescent dye molecule easier to emit a plurality of kinds of luminescence, the luminescent dye molecule may preferably include two or more, more preferably three or more and six or less chromophores or luminophores. When having a plurality of chromophores or luminophores, the luminescent dye molecule may include only one kind of chromophores or luminophores or two or more kinds of chromophores or luminophores. Usually, in the luminescent dye molecule, one chromophore or one luminophore is bonded to one sugar structure in the main chain. Therefore, when the luminescent dye molecule has two or more chromophores or luminophores, the main chain preferably has two or more sugar structures. That is, the number of chromophores or luminophores in the luminescent dye molecule is preferably equal to or smaller than the number of sugar structures in the main chain.

Note that when the number of chromophores or luminophores in the luminescent dye molecule is smaller than the number of sugar structures in the main chain, some of the sugar structures are in a state where no chromophore or luminophore is bonded thereto. Although no other atomic group or the like may be bonded to the sugar structure to which a chromophore or a luminophore is not bonded, a natural base may be bonded thereto, as long as the object and the effects of the present embodiment are not impaired. As used herein, the natural base refers to adenine, guanine, cytosine, thymine, and uracil. The total number of natural bases bonded to the sugar structures is preferably 50% or less, and more preferably 25% or less, with respect to the total number of sugar structures included in the main chain. When the number of the natural bases is 50% or less, association between the luminescent dye molecules is suppressed, and interaction between an object and the luminescent dye molecule is likely to be dominant. In addition, the natural base and the non-natural base are preferably bonded so that the sugar structure is in a β-form.

Here, examples of the chromophore or luminophore that emits fluorescence include a structure derived from fluorescein, rhodamine, boron-dipyrromethene, or the like. Examples of the chromophore or luminophore that emits phosphorescence include a structure derived from an iridium complex, a platinum complex, or the like. Examples of the chromophore or luminophore that exhibits excimer emission include a structure derived from pyrene, anthracene, perylene, or the like. Examples of the chromophore or luminophore that exhibits exciplex emission include a structure derived from pyrene-dimethylaniline or the like. Examples of the chromophore or luminophore that emits thermally activated delayed fluorescence include a structure derived from 4CzIPN, DABNA, or the like. Examples of the chromophore or luminophore that exhibits excited state intramolecular proton emission include a structure derived from hydroxyphenylbenzoxazole or the like. Examples of the chromophore or luminophore that exhibits triplet-triplet annihilation emission include a structure derived from 9,10-diphenylanthracene, rubrene, or the like. Examples of the chromophore or luminophore that exhibits twisted intramolecular charge transfer emission include a structure derived from diaminoanthracene, diaminonaphthalene, or the like. Examples of the chromophore or luminophore that exhibits aggregation-induced emission include a structure derived from tetraphenylethene, hexaphenylsilole, or the like.

Among these, the luminescent dye molecule preferably includes, as the chromophore or luminophore, at least one structure selected from the structure emitting fluorescence, the structure exhibiting excimer emission, and the structure exhibiting exciplex emission, and preferably includes at least the structure emitting fluorescence. Using the luminescent dye molecule that emits fluorescence is advantageous in that it is easy to analyze with various measurement devices.

Furthermore, it is preferable that the luminescent dye molecule exhibits a plurality of types of emissions by irradiation with light having a wavelength of 300 to 400 nm. When the luminescent dye molecule exhibits a plurality of types of emissions by irradiation with light having the wavelength described above, a special light source is not required, whereby the target substance is less likely to be damaged when the target substance is analyzed.

The molecular weight of the luminescent dye molecule is appropriately selected depending on the type of the chromophore and the luminophore of the luminescent dye molecule, the length of the main chain, and the like, and is usually preferably 500 or more and 10,000 or less, more preferably 100 or more and 4,000 or less. When the molecular weight of the luminescent dye molecule is 10,000 or less, the specificity for the target substance becomes moderately low, and it becomes possible to cause the luminescent dye molecule to nonspecifically react with a plurality of sites of the target substance.

### (Method for Producing Luminescent Dye Molecule)

For the luminescent dye molecule, a monomer is prepared in which the chromophore or the luminophore and a phosphate ester are bonded to pentose or hexose. The monomer is polymerized in a desired sequence using a phosphoramidite method with a DNA/RNA synthesizer or the like, by which the luminescent dye molecule can be synthesized. According to such a method, for example, as illustrated in the schematic diagram of FIG. 23, a plurality of types of monomers (three types in FIG. 23) having different kinds of chromophores or luminophores (denoted by A, B, and C in FIG. 23) can be prepared, and a desired number of the monomers can be bonded (three monomers can be bonded in FIG. 23) by changing the order of sequence of the monomer. That is, a wide variety of luminescent dye molecules can be synthesized from a plurality of types of monomers having different types of chromophores or luminophores. In the example illustrated in FIG. 23, 27 types of luminescent dye molecules can be synthesized. By changing the type of the monomer to be used and the number of bonds of the monomer, an enormous number of types of luminescent dye molecules can be synthesized.

### (Effect of Luminescent Dye Molecule)

As described above, the luminescent dye molecule according to the present embodiment has a main chain structure similar to that of a substance existing in the natural world (for example, DNA or RNA). Therefore, the luminescent dye molecule can easily interact with various target substances, and thus, according to the luminescent dye molecule, it is possible to grasp the state of the target substance in detail. In addition, the above-described luminescent dye molecule exhibits a plurality of types of emissions by irradiation with light having a specific wavelength. Therefore, it is possible to obtain a large amount of complicated emission data according to the state of the target substance, and it is possible to analyze the target substance in great detail.

### 2. Analysis Method

An example of the analysis method using the luminescent dye molecule will be described below, but the analysis method using the luminescent dye molecule is not limited thereto.

### (Steps S101 to S105)

FIG. 24 illustrates a flowchart of the analysis method. In the analysis method, one of the luminescent dye molecule and the target substance (hereinafter, also referred to as a “first component”) is placed in a reaction field of a plate that allows the luminescent dye molecule and the target substance to interact with each other (S101, hereinafter, also referred to as a “first component placement step”). Next, first signal information is acquired from the plate on which the first component is placed (S102, also referred to as “first signal information acquiring step”). Then, the other of the luminescent dye molecule and the target substance (hereinafter, also referred to as “second component”) is further placed in the reaction field of the plate from which the first signal information has been acquired (S103, hereinafter, also referred to as “second component placement step”). Then, second signal information is acquired from the plate on which the luminescent dye molecule and the target substance are placed (S104, hereinafter also referred to as a “second signal acquiring step”). Thereafter, the first signal information and the second signal information are compared and analyzed by an analyzer (S105, hereinafter also referred to as an “analysis step”). Thereafter, the first signal information and the second signal information are compared and analyzed (S105, hereinafter, also referred to as an “analysis step”).

The type of the target substance to be analyzed with the analysis method according to the present embodiment is not particularly limited, and for example, the target substance may be a substance whose structure is known or a substance whose structure is unknown. Furthermore, the target substance may be a mixture or the like of various compounds, and may be a substance, a compound, or a composition belonging to any field such as the medical field, the industrial field, or the food field. Examples of the target substance belonging to the medical field include proteins, antibodies, beads with antibodies, and tumor markers. On the other hand, examples of the target substance belonging to the industrial field include metal nanoparticles, carbon nanotubes, magnetic fluid, nanosilica, and crystalline zirconia. Examples of the target substance belonging to the food field include agricultural products and processed products thereof.

### (Second Sensing Device of Emission Data Acquirer 1003)

As another example of the emission data acquirer 1003, a second sensing device can be applied. An organic EL element (OLED) sensor, a microwell sensor with a built-in OLED light source, or the like can be applied. The OLED sensor uses a light emitting layer between electrodes as a reaction field by applying an OLED and emits light with the light-emitting material and an object being allowed to coexist in the light emitting layer. The microwell sensor with a built-in OLED light source uses an OLED as a light source of the microwell and can control desired excitation light. The OLED sensor shines by its own light by current excitation, and thus is advantageous in light emission efficiency and can provide high detectability. Due to such a characteristic, the emission data acquirer 1003 instantaneously acquires a large amount of nanoscale data.

### [Overview of Second Sensing Device]

An example of the second sensing device will be described. The second sensing device described below is a sensing device including at least a pair of electrodes and a receptive layer present between the electrodes, wherein the receptive layer has a detection region in which an analyte and a luminescent compound are dispersed at a molecular level and are present in a thickness direction, the detection region being capable of emitting light.

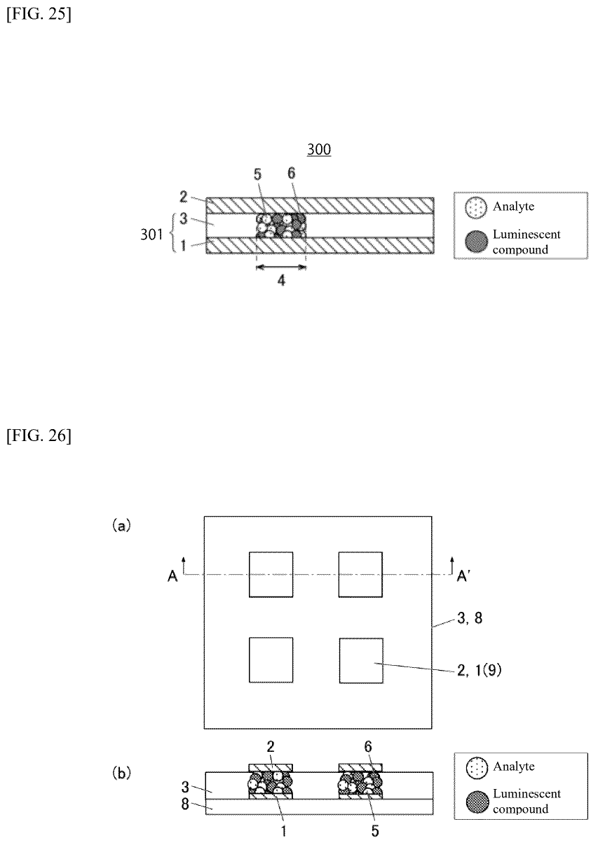

FIG. 25 is a schematic cross-sectional view of the second sensing device.

As illustrated in FIG. 25, a second sensing device 300 includes at least a pair of electrodes 1 and 2, and a receptive layer 3 present between the electrodes 1 and 2, and the receptive layer 3 has a detection region 4 in which an analyte 5 and a luminescent compound 6 are dispersed at a molecular level and present in a thickness direction, the detection region 4 being capable of emitting light.

Specifically, the electrode 1 and the receptive layer 3 (also referred to as an “ink receiving layer”) provided on the electrode 1 constitute a sensing plate 301. The receptive layer 3 of the sensing plate 301 has the detection region 4, and the electrode (also referred to as a “counter electrode”) 2 is provided so as to face the electrode 1.

In the present example, the “detection region” refers to a region in the receptive layer in which the analyte applied to the receptive layer and the luminescent compound are permeated and are present close to each other at a molecular level and which emits light by application of a voltage and is capable of detecting the emitted light. That is, the detection region functions as at least a light emitting layer. Specifically, like a region 4 illustrated in FIG. 25, the detection region refers to an area in which the analyte and the luminescent compound are dispersed at a molecular level and are present in the thickness direction and which can emit light.

In the present example, the wording “dispersed at a molecular level” means that the analyte and the luminescent compound are dispersed in a state where the molecule of the analyte and the molecule of the luminescent compound are not aggregated and are close to each other in isolation or so as to interact with each other, and that the analyte and the luminescent compound are dispersed in a state where a quenching phenomenon does not occur due to the aggregation of the luminescent compound itself even if several molecules are aggregated.

The wording “present in the thickness direction” means that the molecules of the analyte and the luminescent compound are present in the above-described dispersed state in the thickness direction from the upper surface to the lower surface of the receptive layer. Therefore, the state indicated by the wording “present in the thickness direction” is different from a state where the analyte and the luminescent compound are placed in the receptive layer by an amount corresponding to the thickness of a single molecule or a state where only the analyte is present continuously between the upper and lower electrodes.

When the thickness of the detection region described above is within a range of 5 to 500 nm, the analyte and the luminescent compound sufficiently interact with each other. Thus, the detection region having a thickness within the above range provides high emission efficiency and excellent sensing sensitivity. The thickness within the above range is also preferable in that the drive voltage is high and the short-circuit current between the upper and lower electrodes can be suppressed, and the thickness is more preferably within the range of 10 to 100 nm.

The thickness of the detection region can be measured by a stylus profiling system Dektak (manufactured by BRUKER Corporation).

Note that an analyte (not illustrated) is present between the receptive layer and the upper electrode in a region other than the detection region 4.

Note that the detection region also preferably contains a host compound as described in detail later. Furthermore, the receptive layer may have various functional layers such as an electron transport layer and a hole transport layer between the detection region and the electrode.

In the second sensing device, it is preferable that a plurality of the detection regions is present in an in-plane direction of the receptive layer.

FIG. 26 is a schematic cross-sectional view of a sensing device in a case where a plurality of detection regions is present in an in-plane direction of a receptive layer. FIG. 26(a) is a plan view of a sensing element 1, and FIG. 26(b) is a schematic cross-sectional view taken along a line A-A′. Note that reference signs in the drawings are the same as those in FIG. 25 and the like described above, and reference sign 8 and reference sign 9 which are not included in the above-described drawings respectively indicate a substrate and an electrode region (a region where an anode 2 and a cathode 3 overlap each other in plan view). Here, the electrode 1 represents an anode, and the electrode 2 represents a cathode. It is preferable that a plurality of the detection regions is present in the in-plane direction of the receptive layer from the viewpoint that a plurality of types of data can be obtained at a time. FIG. 26 illustrates a case where four detection regions are present at four positions in the in-plane direction of the receptive layer 3.

Further, in the second sensing device, it is preferable that a plurality of types of the analytes is present in the plurality of detection regions or a plurality of types of the luminescent compounds is present in the plurality of detection regions from the viewpoint that a plurality of types of data can be obtained at a time. In particular, in the second sensing device, it is preferable that a plurality of types of the analytes is present in the plurality of detection regions, and a plurality of types of the luminescent compounds is present in the detection regions.

Here, the wording “a plurality of types of analytes or luminescent compounds is present in a plurality of detection regions” means that a plurality of types of analytes or luminescent compounds may be present in a plurality of detection regions or a plurality of types of analytes or luminescent compounds may be present in one detection region.

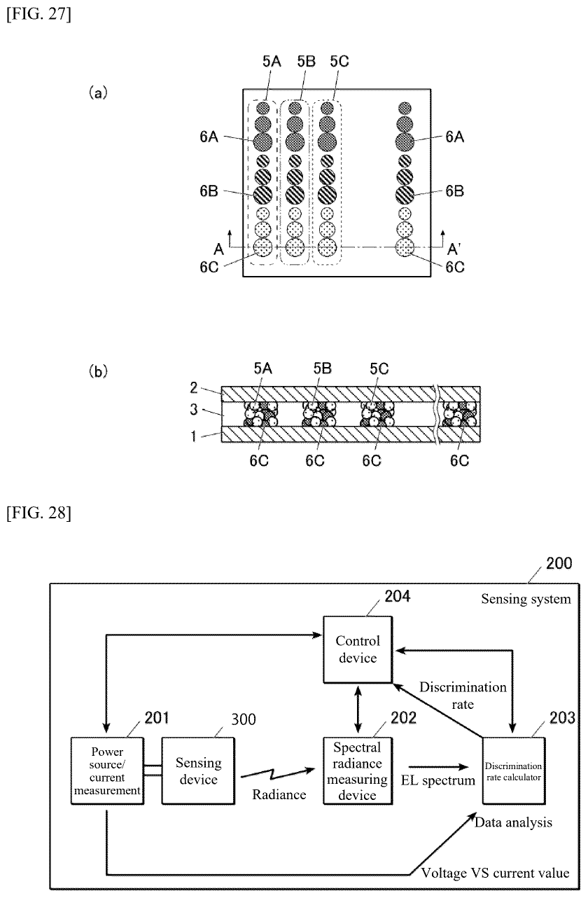

FIG. 27 is a schematic cross-sectional view of a sensing device in a case where a plurality of types of analytes and luminescent compounds are present in a plurality of detection regions.

In the plurality of detection regions, a plurality of types of only the analyte may be present, a plurality of types of only the luminescent compound may be present, or a plurality of types of both the analyte and the luminescent compound may be present. FIG. 27 illustrates a case where three types of analytes 5A, 5B, and 5C and three types of luminescent compounds 6A, 6B, and 6C are used. In FIGS. 27(a), 5A, 5B, and 5C indicate applied areas. For example, three types of analytes and two types of luminescent compounds (a blue phosphorescent compound and a red phosphorescent compound) may be used.

Furthermore, it is preferable that the second sensing device has, in the receptive layer, a reference region where the analyte is not present and only the luminescent compound is present. When the receptive layer includes the reference region in which the analyte is not present and only the luminescent compound is present, the difference between the detection region in which the analyte is present and the reference region is used as a difference value, and thus it is possible to eliminate variations between elements, with the result that it is possible to improve discrimination accuracy.

Further, the receptive layer may have a region having a plurality of types of ratios of the number of molecules of the analyte to the number of molecules of the luminescent compound. Examples of such a configuration include a configuration having a plurality of types of regions where luminescent compounds having different concentrations are applied to analytes having a constant concentration, and a configuration having a plurality of types of regions where luminescent compounds having a constant concentration are applied to analytes having different concentrations. As a result, a plurality of types of detection regions can be formed, and many types of data can be obtained.

Specific examples thereof include a case where luminescent compounds with different concentrations (5 mg/mL and 10 mg/mL) are provided for an analyte with a constant concentration. Usually, the receptive layer is preferably provided as one continuous layer.

The area where the receptive layer is formed is appropriately selected depending on, for example, the type of the analyte onto which an ink is to be dropped.

The receptive layer according to the present example is preferably at least one kind of insulating polymer.

### (Insulating Polymer)

The “insulating property” of the insulating polymer used in the ink receiving layer according to the present example means that the electric resistivity is 1×10⁶ Ω·m or more, preferably 1×10⁸ Ω·m or more, and more preferably 1×10¹⁰ Ω·m or more. When the electric resistivity of the insulating polymer alone is 1×10⁶ Ω·m or more, it is considered that the leakage current flowing in the detection region can be suppressed.

The type of the insulating polymer is not particularly limited as long as the ink receiving layer can be formed. In one embodiment, a polymer having higher stability and having a main chain composed of carbon atoms is used as the insulating polymer.

In order that the ink receiving layer containing the insulating polymer can be formed with a coating method, the insulating polymer is preferably a soluble polymer, and preferably exhibits solubility in an aprotic polar solvent. Specifically, the dissolubility of the insulating polymer in 1 g of N,N-dimethylformamide at 25° C. is preferably 0.5 mg or more, more preferably 1.0 mg or more, and even more preferably 2.0 mg or more.

The type of the insulating polymer is not particularly limited, and examples thereof include: nonionic polymers such as polystyrene, polymethyl methacrylate, polyvinyl alcohol, polyacrylamide, polyvinylpyrrolidone, polyvinylpolypyrrolidone, polyethylene glycol, polymethyl vinyl ether, and polyisopropylacrylamide; cationic polymers such as sodium polyacrylate, sodium polystyrenesulfonate, sodium polyisopropylenesulfonate, polynaphthalenesulfonic acid condensate salts, and polyethyleneimine xanthate salts; anionic polymers such as dimethylaminomethyl (meth)acrylate quaternary salts, dimethyldiallylammonium chloride, polyamidine, polyvinylimidazoline, dicyandiamide-based condensates, epichlorohydrin dimethylamine condensates, and polyethyleneimine; and amphoteric polymers such as dimethylaminoethyl (meth)acrylate quaternary salt-acrylic acid copolymers and Hoffman degradation products of polyacrylamide. Preferably, the insulating polymer is polystyrene or polymethyl methacrylate.

The insulating polymer may contain two or more different repeating units.

The weight-average molecular weight of the insulating polymer is not particularly limited, but is preferably 5×10³ or more, and more preferably 10×10³ or more. The weight-average molecular weight of the insulating polymer is also preferably 1000×10³ or less, more preferably 400×10³ or less. A formulation having a weight-average molecular weight within this range can be used.

### <Analyte>

The analyte is not particularly limited as long as it is a measurement object that can be identified by the second sensing device, and examples thereof include ions, wine, water, and adhesive.

At least the following type of industry and industrial field are assumed as those to which the second sensing device is applicable based on a detection result by the second sensing device.

Examples thereof include production and sales of wine, drinking water, and an adhesive, and the healthcare industry, detection of Na ions and Cl ions in sweat, and detection of Cl ions, Na ions, K ions, and Ca ions in blood since ion components can be identified.

Furthermore, the second sensing device can be used for rainwater, river, small pond, water for breeding tropical fish, soils, countermeasures against salt damage, environment measurement, countermeasures against salt damage, soils, and cultivation management, and also for wastewater treatment/management industries and the like because the second sensing device can discriminate water.

Furthermore, the second sensing device can be used for chemical manufacturing industry such as resin manufacturing because it can identify a very small amount of resin components and solvent components in an adhesive, and can also be used for food processing/manufacturing industry because it can identify a very small amount of mineral components in water and water quality itself.

### <Luminescent Compound>

In the present example, the luminescent compound refers to a fluorescent compound, a delayed fluorescent compound, or a phosphorescent compound.

In the present example, a plurality of types of the luminescent compounds may be used. For example, different phosphorescent compounds may be used in combination, or a phosphorescent compound and a fluorescent compound may be used in combination. Thus, any emission color can be obtained.

In the present example, using a compound that emits blue phosphorescence (blue phosphorescent compound) is preferable, because the sensitivity of the sensing device can be improved.

### (Host Compound)

In the second sensing device, the presence of the host compound around the luminescent compound is preferable, because this configuration facilitates carrier transfer to thereby reduce a voltage. As the host compound, the following conventionally known compounds can be used. Alternatively, not one type but a plurality of types may be used in combination. Use of two or more host compounds enables control of the charge transfer.

Preferable compounds as the conventionally known host compound include a compound which has a hole transporting ability and an electron transporting ability, prevents the wavelength of emitted light from shifting to longer wavelengths, and has a high glass transition temperature (Tg).

Furthermore, the host compound is desirably a low molecular weight host compound. It is known that, when a polymer host compound is used, the drive voltage generally increases (see Japanese Unexamined Patent Publication No. 3-171590). The proportion of the low molecular weight host is preferably 50% or more, more preferably 90% or more of the entire host. When the low molecular weight host compound is 50% or more, the voltage difference can be reduced, and when it is 90% or more, the voltage difference can be almost eliminated. Here, the term “low molecular weight” refers to a molecule having a molecular weight of 2,000 or less.

The host compound is a compound responsible for charge transport to the luminescent compound, and the emission of light from the host compound is not substantially observed in the sensing device.

The host compound may be used alone or a plurality of types of the host compounds may be used in combination. By using a plurality of types of host compounds, the movement of charges can be adjusted, and the efficiency of the sensing device can be increased.

It is preferable that the host compound can be stably present in all active species states of a cation radical state, an anion radical state, and an excited state, and does not cause a chemical change such as decomposition or an addition reaction, from the viewpoint of driving stability. Furthermore, it is preferable that molecules of the charge transporting compound do not move at an angstrom level in the layer with the lapse of time under energization.

### <Layer Configuration of Sensing Device>

The second sensing device includes the pair of electrodes, a receptive layer (also referred to as an “ink receiving layer”) present between the electrodes, and a detection region in the receptive layer. The second sensing device may include, as another layer, an organic functional layer such as a hole injection layer, a hole transport layer, an electron transport layer, or an electron injection layer in the receptive layer.

As a representative configuration example of the second sensing device, the following configuration examples can be given, but the configuration is not limited thereto.
(i) anode/receptive layer (detection region)/cathode
(ii) anode/receptive layer (detection region/electron transport layer)/cathode
(iii) anode/receptive layer (hole transport layer/detection region)/cathode
(iv) anode/receptive layer (hole transport layer/detection region/electron transport layer)/cathode
(v) anode/receptive layer (hole transport layer/detection region/electron transport layer/electron injection layer)/cathode
(vi) anode/receptive layer (hole injection layer/hole transport layer/electron blocking layer/detection region/hole blocking layer/electron transport layer)/cathode

Note that any one of the hole injection layer, the hole transport layer, the electron blocking layer, the detection region, the hole blocking layer, and the electron transport layer may be provided not inside the receptive layer but between a receptive layer and an electrode (anode or cathode) located outside.

The “electron transport layer” according to the present example is a layer having a function of transporting electrons, and in a broad sense, the electron injection layer and the hole blocking layer are also included in the electron transport layer. Further, it may include a plurality of layers.

The “hole transport layer” according to the present example is a layer having a function of transporting holes, and in a broad sense, the hole injection layer and the electron blocking layer are also included in the hole transport layer. Further, it may include a plurality of layers.

### [Method for Manufacturing Sensing Device]

A method for manufacturing a second sensing device includes the steps of: applying the analyte to a receptive layer formed on one electrode; applying the luminescent compound to a portion to which the analyte has been applied; and providing the other electrode so as to face the one electrode.

In addition, it is preferable that the step of applying the analyte is a step using an inkjet printing method. With this configuration, a large amount of analytes can be easily applied, whereby a measurement field (sensing place) can be mass-produced.

Since it is sufficient that the analyte and the luminescent compound are finally dispersed in proximity to each other at a molecular level, the order of application of the analyte and the luminescent compound may be reversed. Further, they may be mixed by being ejected almost simultaneously by an ink jet, or a solution in which both are mixed in advance may be ejected.

Further, it is preferable that the step of providing the other electrode is a step of bonding the other electrode. This configuration can miniaturize the device without requiring a vacuum process such as vapor deposition.

### [Sensing System]

A sensing system associated with the second sensing device includes at least the sensing device described above, a power source for supplying a carrier for exciting a luminescent compound in the sensing device by current, a spectral radiance measuring device for measuring radiance from the luminescent compound and outputting an emission spectrum, and a discrimination rate calculator for calculating a discrimination rate using the emission spectrum.

FIG. 28 is a schematic diagram illustrating the configuration of a sensing system according to the first embodiment of the present invention.

As illustrated in FIG. 28, a sensing system 200 includes a sensing device 300, a power source 201, a spectral radiance measuring device 202, a discrimination rate calculator 203, and a control device 204.

The sensing device is as described above, and therefore, the description thereof is omitted.

The power source supplies a carrier for exciting the luminescent compound in the sensing device by current, and may be a DC power source or an AC power source, but is preferably an AC power source. In addition, it is preferable that the AC power source has, for example, a frequency of about 1 Hz to 100 MHz, an offset voltage of about 0 to 20 V, and an amplitude voltage of about 0 to 20 V.

The power source preferably has a current measurement function, and outputs a measured voltage-current characteristic to the discrimination rate calculator.

In addition, the control device outputs the number of times of voltage supply, voltage supply time, and AC voltage application conditions to the discrimination rate calculator.

Such a power source may be any commercially available device that can apply the above-described frequency, offset voltage, and amplitude voltage, and for example, power sources such as 6243 DC voltage current source/monitor (manufactured by ADC Corporation) and Function Generator FG300 (manufactured by Yokogawa Electric Corporation) can be used. It is sufficient that the current measurement device can measure a current of about 1 nA to 100 mA, and for example, Source Meter 2400 (manufactured by Keithley Instruments) can be used.

The spectral radiance measuring device outputs the measured data regarding the radiance and emission spectrum of light emitted from the detection region of the sensing device to the discrimination rate calculator.

As the spectral radiance measuring device, for example, a spectroradiometer CS-2000 (manufactured by Konica Minolta, Inc), a hyperspectral camera NH-6-700 (manufactured by Eva Japan Co., Ltd.), or the like is preferably used.

The discrimination rate calculator calculates a discrimination rate from the voltage application condition output from the control device, the current characteristic output via the control device, and the emission spectrum output from the spectral radiance measuring device.

The discrimination rate calculator calculates a discrimination rate based on the number of times of voltage supply and the voltage supply time output from the control device.

The discrimination algorithm by the discrimination rate calculator is preferably any one of principal component analysis (PCA), cluster analysis (hierarchical clustering, k-means clustering, clustering using Gaussian mixture model), linear discriminant analysis (LDA), partial least squares method (PLS regression), decision tree, random forest, bootstrap forest, neural network, neural boosting, K-nearest neighbor algorithm, Naive Bayes, support vector machine (SVM), nominal logistic (multi-logit), and generalized regression (Ridge, lasso), or a combination thereof. These will be described below.

### <Analysis Method>

The statistical analysis and the analysis in machine learning can be performed by principal component analysis (PCA), cluster analysis (hierarchical clustering, k-means clustering, and clustering using Gaussian mixture model), linear discriminant analysis (LDA), partial least squares method (PLS regression), or the like, or a combination thereof, using statistical analytical software JMP 16.2 or JMP pro 16.2 available from SAS Institute Japan Ltd. In addition, a decision tree, a random forest, a bootstrap forest, a neural network, a K-nearest neighbor algorithm, Naive Bayes, a support vector machine (SVM), nominal logistic (multi-logit), generalized regression (Ridge, lasso), or the like may be used.

In a case where the number of samples is about 50 or less, the explanatory variable is 100 or more, and the variable is spectral data as in an example described later, PCA or PLS regression which is linear regression using a principal component that has been subjected to dimensionality reduction, and LDA using a principal component (a wide data method in JMP) are more preferable from the viewpoints of suppression of overtraining and the robustness of the result. However, depending on the data set, the above-described analysis methods other than these may be effective.

### <Linear Discriminant Analysis (LDA)>

Regarding the linear discriminant analysis (LDA), JMP 16.2 has a stepwise method for arbitrarily selecting an explanatory variable and a wide-data method for performing dimensionality reduction on a principal component, and either of the methods can be applied. The wide-data method is preferable from the viewpoints of a reduction in calculation time and the suppression of overtraining described above.

### <Evaluation of Discrimination Performance>

For the evaluation of the discrimination performance, all pieces of the sample data were classified into training data and verification data at an arbitrary ratio, and linear discriminant analysis (LDA) was performed to calculate the discrimination rate (accuracy, predictive value), the error rate, and the entropy R square. For the calculation of the verification result using the verification data, it is preferable to use a hold-out method using any or random pair of verification datasets, or k-fold cross validation in which all pieces of sample data are divided into k subsets and cross validation is performed using the k subsets.

In addition, as a result of the k-fold cross validation, it is preferable to display an average value of k discrimination models, a discrimination model having the best statistic (having the smallest error from the average) among k discrimination models, or a discrimination model having the smallest difference in discrimination rate or entropy R square between training and verification.

The control device controls a condition of applying DC or AC voltage to the sensing device, the number of times of voltage supply, and the voltage supply time, and also controls the timing of the measurement of radiance by the spectral radiance measuring device and current measurement by the power source.

Further, the control device integrally controls the entire operation of the sensing system, and includes a memory, a controller, a calculation descriptor construction section, and the like. Furthermore, a user can input measurement processing conditions and the like to the control device with a keyboard and a mouse.

The discrimination rate calculator may be integrated with the control device.

Next, an example of processing performed by the sensing system configured as described above will be described.

First, the control device 204 drives the power source 201 to apply a predetermined voltage from the power source 201 to the sensing device 300. Thus, the detection region of the sensing device 300 emits light. Here, the condition of applying DC or AC voltage supplied to the sensing device 300 and the data regarding the number of times of voltage supply and the voltage supply time are output to the discrimination rate calculator 203.

Next, the control device 204 drives the spectral radiance measuring device 202 to measure the radiance due to light emission in the detection region. The measured emission spectrum is output to the discrimination rate calculator 203.

Next, the control device 204 drives the discrimination rate calculator 203 to calculate the discrimination rate based on the output emission spectrum, the condition of applying DC or AC voltage output from the power source 201, the number of times of voltage supply, and the voltage supply time. The calculated discrimination rate is displayed on a display (not illustrated) of the control device 204.

Note that the control device preferably controls the driving of the power source, the timing of measuring the radiance by the spectral radiance measuring device, and the timing of measuring a current by the power source so that the number of times of voltage supply and the voltage supply time input by the user with the keyboard and the mouse can be obtained.

As the materials and constituent elements of the first and second sensing devices described above, known materials and constituent elements related to organic electroluminescent elements can be used without limitation.

### Other Modification Examples

Regarding the configurations of the information processing apparatus, and the like described above, the main configuration has been described for describing the features of the above-described embodiments. The present invention is not limited to the above-described configuration and can be variously modified within the scope of the claims. In addition, a configuration included in a general information processing apparatus or the like is not excluded.

The embodiments and Examples may be applied in combination. For example, the first to third embodiments may be applied in combination with each other, and these combinations may be applied to Examples 1 to 6. In addition, the information processing apparatus 10 (or 10b or 10c) may output the prediction result (the characteristic and the like of the object) obtained by the predictor 120 in step S12, or the like to the user. For example, the information processing apparatus 10 displays the prediction result on a display or transmits the prediction result to a terminal device (PC) set in advance. The user who has received the prediction result can recognize in advance to what extent the predicted characteristic and the like of the final product deviate from the characteristic and the like to be realized of the final product set in advance, or to what extent the predicted characteristic and the like are close to the target.

In addition, means and methods for performing the various processes in the information processing apparatus 10, 10b, or 10c according to the above-described embodiments can be implemented by any of a dedicated hardware circuit and a programmed computer. The program described above may be provided by, for example, a computer-readable recording medium such as a USB memory or a digital versatile disc (DVD)-ROM, or may be provided online via a network such as the Internet. In this case, the program recorded on the computer-readable recording medium is usually transferred to and stored in a storage such as a hard disk. The above program may be offered as standalone application software or may be incorporated, as a function of an apparatus, into software for the apparatus.

The present application is based on Japanese Patent Application (Japanese Patent Application No. 2023-057129) filed on Mar. 31, 2023, the disclosure content of which is incorporated by reference in its entirety.

### REFERENCE SIGNS LIST

10 information processing apparatus
110 descriptor construction section
120 predictor
131 first process parameter adjuster
132 second process parameter adjuster
141 first storage
142 second storage
143 third storage
100, 1000 acquirer
101 first data acquirer
102 second data acquirer
103 third data acquirer
1001 ultrasound data acquirer
1002 electromagnetic-wave data acquirer
1003 emission data acquirer
105 characteristic acquirer

1. An information processing apparatus comprising, in a design and manufacturing process of a product or a material having a plurality of predetermined characteristics:
a descriptor construction section that constructs a descriptor based on data obtained by measuring information regarding a structure and/or a property of an object in the design and manufacturing process under a plurality of predetermined measurement conditions; and
a predictor that predicts a plurality of characteristics of a final product of the design and manufacturing process based on the descriptor.

2. The information processing apparatus according to claim 1, wherein the object is a final product obtained as an output result of the design and manufacturing process, an intermediate product generated during the design and manufacturing process, and/or a starting material input to the design and manufacturing process.

3. The information processing apparatus according to claim 1, wherein the plurality of measurement conditions includes at least a measurement condition in which spatial or temporal measurement regions and/or spatial or temporal measurement scales are different.

4. The information processing apparatus according to claim 3, wherein the plurality of measurement conditions is selected depending on the predetermined characteristics of the object.

5. The information processing apparatus according to claim 3, wherein the plurality of measurement conditions includes at least a first measurement condition having a predetermined first measurement region and a first measurement scale, and a second measurement condition set based on the first measurement condition, and
the second measurement condition has a second measurement region smaller than the first measurement region or a second measurement scale smaller than the first measurement scale.

6. The information processing apparatus according to claim 5, wherein one of data acquired under the first measurement condition and data acquired under the second measurement condition is information regarding the structure of the object, and the other is information regarding the property of the object.

7. The information processing apparatus according to claim 3, wherein measurement timings under the plurality of measurement conditions are simultaneous with each other or have a certain temporal relation with each other.

8. The information processing apparatus according to claim 7, wherein the measurement timings are determined to be synchronized with a change in position or a change in place of the object in the design and manufacturing process as the certain temporal relation.

9. The information processing apparatus according to claim 5, wherein, under the first and second measurement conditions, the first and second measurement regions have a predetermined spatial relation with each other.

10. The information processing apparatus according to claim 1, wherein the predictor predicts a plurality of characteristics of the final product from the entire or a part of the descriptor, using a first trained model that outputs characteristics of the object based on the descriptor.

11. The information processing apparatus according to claim 1, further comprising a process parameter adjuster that, using a second trained model that outputs a parameter related to the design and manufacturing process based on the descriptor, adjusts the parameter based on the entire or a part of the descriptor.

12. The information processing apparatus according to claim 3, wherein the plurality of measurement conditions uses frequencies and/or signal lengths different from each other.

13. The information processing apparatus according to claim 12, wherein the plurality of measurement conditions is executed by a plurality of measurement means, and
the plurality of measurement means includes at least any two of an acoustic sensor, an electromagnetic-wave sensor, and a light emission sensor.

14. A characteristic prediction method comprising, in a design and manufacturing process of a product or a material having a plurality of predetermined characteristics:
a step (a) of constructing a descriptor based on data obtained by measuring information regarding a structure and/or a property of an object in the design and manufacturing process under a plurality of predetermined measurement conditions; and
a step (b) of predicting a plurality of characteristics of a final product of the design and manufacturing process based on the descriptor.

15. A computer-readable recording medium storing a control program for causing a computer to execute processing comprising, in a design and manufacturing process of a product or a material having a plurality of predetermined characteristics:
a step (a) of constructing a descriptor based on data obtained by measuring information regarding a structure and/or a property of an object in the design and manufacturing process under a plurality of predetermined measurement conditions; and
a step (b) of predicting a plurality of characteristics of a final product of the design and manufacturing process based on the descriptor.
